SEQUENCE LISTING (4 sequences)

SEQ ID NO: 1 - DNA, 26 bp, Homo sapiens
ctccatctcactggataatggtactg

SEQ ID NO: 2 - DNA, 26 bp, Homo sapiens
gacactcataaagagaagtgtggacc

SEQ ID NO: 3 - DNA, 22 bp, Homo sapiens
ctcgcgctactctctctttctg

SEQ ID NO: 4 - DNA, 23 bp, Homo sapiens
gcttacatgtctcgatcccactt

(19) United States
(12) Patent Application Publication
(10) Pub. No.: US 2002/0155440 A1
(43) Pub. Date: Oct. 24, 2002

(54) Using overexpression of laminin alpha 4 subunit as a diagnostic and prognostic indicator of malignant tumors

(76) Inventors: Julia Y. Ljubimova, Studio City, CA (US); Alexander V. Ljubimov, Studio City, CA (US); Keith L. Black, Los Angeles, CA (US)

Correspondence Address: Edward G. Poplawski, Esq., SIDLEY & AUSTIN, 555 West Fifth Street, Los Angeles, CA, 90013-1010, US

(21) Appl. No.: 09/741,550

(22) Filed: Dec. 19, 2000

(51) Int. Cl. C12Q 1/68; G01N 33/574

(52) U.S. Cl. 435/6; 435/7.23

(57) ABSTRACT

Disclosed is a method of diagnosing the presence of a malignant tumor, including a glioma, in a human subject, which involves detecting overexpression of laminin α4 subunit protein or laminin α4-specific MRNA, compared to the expression level in a normal tissue control. Also disclosed are a method of predicting the recurrence of a malignant tumor in a human subject from whom a malignant tumor has been resected and a method of classifying the grade of a malignant tumor, such as a glial tumor, based on a molecular classification.

### BACKGROUND OF THE INVENTION

Throughout the application various publications are referenced in parentheses. The disclosures of these publications in their entireties are hereby incorporated by reference in the application in order to more fully describe the state of the art to which this invention pertains.

1. Field of the Invention

This invention relates to the medical arts. In particular, it relates to a method for predicting, detecting and classifying malignant tumors.

2. Discussion of the Related Art

Malignant tumor growth, progression, and metastasis are largely dependent on neovasculature for access to a steady supply of nutrients and for the removal of wastes. It is also apparent that during the transition from mid-late dysplasia, an “angiogenic switch” is activated; and changes in tissue angiogenic phenotype probably precede the histological tissue transition to malignancy. (Hanahan, D. and Folkman, J., Patterns and emerging mechanisms of the angiogenic switch during tumorigenesis, Cell. 86:353-64 [1996]). Pathologic neovascularization, i.e., the proliferation or development of new blood vessels by the process of angiogenesis, is, thus, essential for the growth and spread of primary, secondary and metastatic malignant tumors.

It is known that certain properties of new capillaries and arterioles of the neovasculature in solid tumors differ from those of normal vasculature. (J. Denekamp et al., Vasculature and microenvironmental gradients: the missing links in novel approaches to cancer therapy?, Adv. Enzyme Regul. 38:281-99 [1998]). Neovasculature induced by angiogenic factors from malignant cells was reported to possess altered pharmacological reactivity to some vasoconstricting agents, compared with neovasculature that was not induced by neoplastic cells. this result indicates that neovasculature is likely to have a different molecular profile from normal vasculature. (S. P. Andrade and W. T. Beraldo, Pharmacological reactivity of neoplastic and non-neoplastic associated neovasculature to vasoconstrictors, Int. J. Exp. Pathol. 79(6):425-32 [1998]).

Several cytokines and growth factors, including basic fibroblast growth factor (bFGF) and vascular endothelial growth factor (VEGF) modulate angiogenesis in vivo. (Bikfalvi, A. et al., Biological roles of fibroblast growth factor-2, Endocr. Rev. 18:26-45 [1997]; Ferrara, N. and Davis-Smyth, T., The biology of vascular endothelial growth factor, Endocr Rev 18:4-25 [1997]). bFGF, VEGF, and other factors are also significantly associated with intratumoral neovascularization. (Expression of the angiogenic factors vascular endothelial cell growth factor, acidic and basic fibroblast growth factor, tumor growth factor-1, platlet-derived endothelial cell growth factor, placenta growth factor, and pleiotrophin in human primary breast cancer and its relation to angiogenesis, Cancer Res. 57:963-69 [1997]; Linderholm, B. et al., Vascular endothelial growth factor is of high prognostic value in node-negative breast carcinoma, J. Clin. Oncol. 16:3121-28 [1998]).

Among the most highly “vascular” of malignant tumors are the glial tumors. This means that within the glial tumors the ratio of neovascular tissue to other cellular tissue is relatively high, compared to normal tissue and most other malignant tumor types. The glial tumors, or gliomas, comprise the majority of primary malignant brain tumors. Gliomas are commonly classified into four clinical grades, with the most aggressive or malignant form of glioma being glioblastoma multiforme (GBM; also known as astrocytoma grade IV), which usually kills the patient within 6-12 months. (Holland, E. C. et al., Combined activation of Ras and Akt in neural progenitors induces glioblastoma formation in mice, Nat. Genet. 25(1):55-57 [2000]; Tysnes, B. B et al, Laminin expression by glial fibrillary acidic protein positive cells in human gliomas, Int. J. Dev. Neurosci. 17(5-6):531-39 [1999]). Despite a wealth of molecular biological, biochemical and morphological information that is available today on gliomas, the prognosis with treatment has not significantly changed in the last two decades and remains among the worst for any kind of malignancy. (E.g., Shapiro, W. R., Shapiro, J. R., Biology and treatment of malignant glioma, Oncology 12:233-40 [1998]; Thapar, K. et al., Neurogenetics and the molecular biology of human brain tumors, In: Brain Tumors, Edit. Kaye A H, Laws E R, pp.990. [1997]).

GBM tumors are characterized by rapid cell growth and extensive invasion into the surrounding normal brain tissue. GBM tumors are difficult to remove surgically and typically recur locally at the site of resection, although metastases also may occur within the central nervous system. Tumor cell movement within the central nervous system is a complex process that involves tumor cell attachment to the extracellular matrix (ECM) via cell surface receptors, degradation of the ECM by proteolytic enzymes, including serine proteases and matrix metalloproteinases, and subsequent tumor cell locomotion. (Tysnes et al. [1999]; MacDonald, T. J. et al., Urokinase induces receptor mediated brain tumor cell migration and invasion, J. Neurooncol. 40(3):215-26 [1998]; Mäenp\ddot{aa}, A. et al., Lymphocyte adhesion molecule ligands and extracellular matrix proteins in gliomas and normal brain: expression of VCAM-1 in gliomas, Acta Neuropathol. (Berl.) 94(3):216-25 [1997]). Thus, malignant gliomas overexpress members of the plasminogen activator system and characteristically invade by migrating on ECM-producing white matter tracts and blood vessel walls. (Tysnes et al. [1999]; Colognato, H. and Yurchenco, P. D., Form and function: the laminin family of heterotrimers, Dev. Dyn. 218(2):213-34 [2000]).

Several ECM components have been proposed as possible key molecules for tumor invasiveness, including collagens (e.g., types I, III, and IV), fibronectins, tenascins, vitronectin, osteopontin, thrombospondins, chondroitin sulfate proteoglycans, hyaluronic acid, and laminins. (e.g., Kulla, A. et al., Tenascin expression patterns and cells of monocyte lineage: relationship in human gliomas, Mod. Pathol. 13(1):56-67 [2000]; Zhang, H. et aL, Expression of a cleaved brain-specific extracellular matrix protein mediates glioma cell invasion In vivo, J. Neurosci.18(7):2370-76 [1998]; Van Aken, M. et al., Detection of complexes which include basement membrane components as diagnostic of cancer and other diseases, U.S. Pat. No. 5,591,830; Kimura, S. et al., Quantitative determination of tenascin as glioma marker, U.S. Pat. No. 5,436,132;).

The laminins are a family of heterotrimeric glycoproteins, each comprised of an alpha (α), a beta (β), and a gamma (γ) chain (or subunit) in an approximately cruciform orientation, that provide an integral part of the ECM structural scaffolding of basement membranes in almost every animal tissue. (Colognato, H. and Yurchenco, P. D., Form andfunction: the laminin family of heterotrimers, Dev. Dyn. 218(2):213-34 [2000]). Twelve isoforms of laminin are known containing distinctive combinations of subunits. (Miner, J. H., Renal basement membrane components, Kidney International 56:2016-2024 [1999]; see also, Geri, M. et al., Monoclonal antibodies for selective immunological determination of high molecular weight, intact laminin forms in bodyfluids, U.S. Pat. No. 5,811,268).

The laminins can self-assemble, bind to other extracellular matrix macromolecules and have unique and shared cell interactions mediated by integrins, dystroglycans, and other receptors. Through these intermolecular interactions laminins significantly contribute to cell differentiation and development, cell shape and movement, maintenance of tissue phenotypes, and promotion of tissue survival. (E.g., Ringelmann, B. et al., Expression of laminin α1, α2, α4, and α5 chains, fibronectin, and tenascin-C in skeletal muscle of dystrophic 129ReJ dy/dy mice, Exp. Cell. Res. 246(1):165-82 [1999]; Ritchie, C. K. et al., Integrin involvement in glioblastoma multiforme: Possible regulation by NF-kappaB, J. Cell. Physiol. 184(2):214-21 [2000]).

Laminin-1 protein was detected in cerebrovascular tissue abnormalities. (Kilic T. et al., Expression of structural proteins and angiogenic factors in cerebrovascular anomalies, Neurosurgery 46(5):1179-91; discussion 1191-92 [2000]). Expression of laminin-1 and laminin-2 has been detected immunohistochemically in the basal lamina of tumor blood vessels, and substantial punctate deposits of laminin-1 were co-localized with the astroglial marker glial fibrillary acidic protein in non-vascular tissue comprising human glioblastoma cells, especially in the confrontation zone between normal and tumor tissue. (Tysnes, B. B et al., Laminin expression by glial fibrillary acidic protein positive cells in human gliomas, Int. J. Dev. Neurosci. 17(5-6):531-39 [1999]; Toti, P. et al., Expression of laminin 1 and 2 in brain tumor vessels. an immunohistochemical study, J. Submicroscop. Cytol. Pathol. 30(2):227-30 [1998]; see also, Bartus, R. T. et al., Evidence that Cereport's ability to increase permeability of rat gliomas is dependent upon extent of tumor growth: implications for treating newly emerging tumor colonies, Exptl. Neurol. 161:234-44 [2000]) While laminin-specific receptors α1β1, α2β1, and α3β1 integrins (adhesion molecules) are present in normal astrocytes, laminin α6β4 integrin, which binds laminin-1, laminin-2, and laminin-5, was differentially overexpressed in human astrocytomas and rat gliomas. (Previtali, S. C. et al., Laminin receptor alpha6beta4 integrin is highly expressed in ENU-induced glioma in rat, Glia 26(1):55-63 [1999]; see also, Ruoslahti, E. I. et al., Adhesion receptor for laminin and its use, U.S. Pat. No. 5,180,809).

Jaffey et al. immunohistochemically detected the presence of extracellular depositions of extracellular matrix proteins collagen IV and laminin-1 in resected malignant gangliogliomas and suggested that they are related to both to perivascular inflammation and the relatively slow proliferation and non-invasiveness of malignant gangliogliomas compared to astrocytomas. (Jaffey P B et al., The clinical significance of extracellular matrix in gangliogliomas, Neuropathol. Exp. Neurol. 55(12):1246-52 [1996]). Astrocytomas lacked these deposits of collagen IV and laminin-1, except in vascular basement membranes and pial membranes.

In contrast, laminin a4 subunit, particular to laminin-8, laminin-9 (Miner, J. H. [1999]), and laminin-14 (Libby, R. T. et al., Laminin expression in adult and developing retinae: Evidence of two novel CNS laminins, J. Neurosci. 2000;20:6517-6528 [2000]), has not been reported in association with any cancer cells or neoplastic tissue types. Laminin α4 chain is found both in adults and during development, in cardiac, skeletal and smooth muscle fibers, vascular endothelium, lungs, synapses, peripheral nerves and in blood cells including monocytes, erythromegakariocytes, and platelets (Miner et al., The laminin alpha chains: expression, developmental transitions, and chromosomal locations of α1-5, identification of heterotrimeric laminins 8-11, and cloning of a novel α3 isoform, J. Cell. Biol. 137:685-701 [1997]; McDonald et al. [1998]; Geberhiwot, T. et al., Erythromegakaryocytic cells synthesize laminin-8 (α4β1γ1y), Exp. Cell. Res. 2000 254:189-195 [2000]; Pedraza, C. et al., Monocytic cells synthesize, adhere to, and migrate on laminin-8, J. Immunol. 165(10):5831-5838 [2000]; Geberhiwot, T. et al., Blood platelets contain and secrete laminin-8 (α4β1γ1) and adhere to laminin-8 via α6,β1 integrin, Exp. Cell. Res. 253:723-732 [1999]; Sunderland, W. J. et al., The presynaptic calcium channel is part of a transmembrane complex linking a synaptic laminin (α4β2γ1) with non-erythroid spectrin, J. Neurosci. 2000;20: 1009-19 [2000]; Ringelmann B. et al., Expression of laminin alpha1 , alpha2, alpha4, and alpha5 chains, fibronectin, and tenascin-C in skeletal muscle of dystrophic 129ReJ dy/dy mice, Exp Cell Res 246(1):165-82 [1999]). The strongest expression of this chain was detected in small and large intestine, smooth and skeletal muscle, placenta, liver, heart, lung and ovary. At the same time, only weak expression was observed in pancreas, testis, prostate, spleen, kidney and brain (Miner et al. [1997]). Alpha 4 chain-containing laminins apparently use integrin α6β1 as their major cell surface receptor. (Sorokin, L. M. et al., Laminin α4 and integrin α6 are upregulated in regenerating dy/dy skeletal muscle: Comparative expression of laminin and integrin isoforms in muscles regenerating after crush injury. Exp Cell Res 2000 256:500-514., Kortesmaa, J. et al., Recombinant laminin-8 (α1β1γ1). Production, purification, and interactions with integrins, J. Biol. Chem. 275:14853-59 [2000]; Talts, J. F. et al., Structural and functional analysis of the recombinant G domain of the laminin α4 chain and its proteolytic processing in tissues, J. Biol. Chem. 275:35192-99 [2000]).

Recent studies have also demonstrated overexpression in gliomas of c-myc and c-met oncogenes, CD44, ICAM-1, CD58 (LFA-3), and smooth muscle actin. Other factors that have been associated with the process of tumor invasion include: matrix metalloproteinase (MMP)-2 and tissue inhibitor of MMP (TIMP)-2 (e.g., Beliveau, R. et al., Expression of matrix metalloproteinases and their inhibitors in human brain tumors, Ann. N.Y. Acad. Sci. 886:236-39 [1999]), transcription factors Sp1, Sp3, and AP-2 (Vince, G. H. et al., Heterogeneous regional expression patterns of matrix metalloproteinases in human malignant gliomas, Int. J. Dev. Neurosci. 17(5-6):437-45 [1999]; Qin, H. et al., The transcription factors Sp1, Sp3, and AP-2 are required for constitutive matrix metalloproteinase-2 gene expression in astroglioma cells, Biol. Chem. 1999, 274:29130-29137 [1999]), the intermediate filament protein vimentin (Farr-Jones, M. A. et al., Improved technique for establishing short term human brain tumor cultures, J. Neurooncol. 43:1-10.39 [1999]), and a number of growth factors. Some of the growth factors that have previously been asociated with tumor growth and progression include: vascular endothelial growth factor (VEGF), human renal cell carcinoma antigen RAGE-1, epidermal growth factor receptor, insulin like growth factor (IGF)-II, transforming growth factor (TGF)-α and TGF-β, fibroblast growth factor (FGF-2), and granulin. (E.g., Neumann, E. et al., Heterogeneous expression of the tumor-associated antigens RAGE-1, PRAME, and glycoprotein 75 in human renal cell carcinoma: candidates for T-cell-based immunotherapies?, Cancer Res. 58:4090-4095 [1998]; Melino, G. et al., IGF-II mRNA expression in LI human glioblastoma cell line parallels cell growth, Neurosci. Lett. 144:25-28 [1992]; Helle, S. I. et al., Influence of treatment with tamoxifen and change in tumor burden on the IGF-system in breast cancer patients, Int. J. Cancer 69:335-339 [1996]; Jennings, M. T. et al., The role of transforming growth factor beta in glioma progression, Neurooncol. 36:123-140 [1998]; Liau, L. M. et al., Identification of a human glioma-associated growth factor gene, granulin, using differential immuno-absorption, Cancer Res. 60(5):1353-60 [2000]).

Chloride channels and chromosomal amplifications and deletions specific to glial-derived tumor cells have also been targeted for diagnostic and therapeutic purposes. (E.g., Ullrich, N. et al., Method of diagnosing and treating gliomas, U.S. Pat. No. 6,028,174; Ullrich, N. et al., Method of diagnosing and treating gliomas, U.S. Pat. No. 5,905,027; Feuerstein, B. G. et al., Glioma-associated nucleic acid probes, U.S. Pat. No. 5,994,529).

Typically, previous studies of glioma markers have been conducted detecting one or a few genes or proteins at a time, despite awareness in the art that families or cascades of genes were actually involved. The development of sensitive nucleic acid microarray detection techniques and analytical methods have recently made it possible to detect gene copy numbers or coordinated gene expression for a large number of different genes simultaneously and thus derive extensive gene expression profiles under various physiological conditions. (E.g., Carulli, J. P. et al., High throughput analysis of differential gene expression, J. Cell Biochem. Suppl. 30-31:286-96 [1998]; Scherer, S., Quantitative methods, systems and apparatuses for gene expression analysis, WO9958720A1; Gerhold, D. et al., DNA chips: promising toys have become powerful tools, Trends Biochem. Sci. 24(5):168-73 [1999]; Duggan, D. J. et al., Expression profiling using cDNA microarrays, Nat. Genet. 21(1 Suppl): 10-14 [1999]; Erlander, M. G. et al., Method for generating gene expression profiles, WO028092A1; Nelson, P. S. et al., Comprehensive analysies of prostate gene expression: convergence of expressed sequence tag databases, transcript profiling and proteomics, Electrophoresis 21(9):1823-31 [2000]; De Benedetti, V. M. et al., DNA chips: the future of biomarkers, Int. J. Biol. Markers 15(1):1-9 [2000]; Bradley, A. et al., Chemically modified nucleic acids and methods for coupling nucleic acids to solid support, U.S. Pat. No. 6,048,695; Lockhart, D. J. et al., Expression monitoring by hybridization to high density oligonucleotide arrays, U.S. Pat. No. 6,040,138; Dehlinger, P. J., Position-addressable polynucleotide arrays, U.S. Pat. No. 5,723,320; Pinkel, D. et al., Comparative fluorescence hybridization to nucleic acid arrays, U.S. Pat. No. 5,830,645).

This nucleic acid array technology has been applied to the diagnosis and/or treatment monitoring of various disease states, including some malignancies, such as breast, ovarian, cervical, pancreatic, and prostatic cancers, rhabdomyosarcoma, lymphoma, and leukemia. (E.g., Leighton, S. B. et al., Tumor tissue microarrays for rapid molecular profiling, WO9944603B1; Augenlicht, L., Method of detecting pathological conditions, U.S. Pat. No. 4,981,783; Vogelstein, B. et al., Gene expression profiles in normal and cancer cells, WO9853319A3; Augenlicht, L., Method for detecting pathological conditions, U.S. Pat. No. 4,981,783, Stoughton, R., Methods of monitoring disease states and therapiesw using gene expression profiles, WO9966024C2; Pinkel, D. et al., Array-based detection of genetic alterations associated with disease, U.S. Pat. No. 6,066,453; Khan, J. et al., Expression profiling in cancer using cDNA microarrays, Electrophoresis 20(2):223-29 [1999]; Gress, T. M. et al., A pancreatic cancer-specific expression profile, Oncogene 13(8): 1819-30 [1996]; Elek, J. et al., Microarray-based expression profiling in prostate tumors, In Vivo 14(1): 173-82 [2000]; Khan, J. et al., Gene expression profiling of alveolar rhabdomyosarcoma with cDNA microarrays, Cancer Res. 58(22):5009-13 [1998]; Ono, K. et al., Identification by cDNA microarray of genes involved in ovarian carcinogenesis, Cancer Res. 60:5007-11 [2000]; Shim, C. et al., Profiling of differentially expressed genes in human primary cervical cancer by complementary DNA expression array, Clin. Cancer Res. 4(12):3045-50 [1998]). None of these methods has described expression of laminin-8 or its α4 subunit as a marker for malignancy or neovascular angiogenesis.

Attempts to find diagnostically and prognostically useful gene expression profiles of gliomas with nucleic acid array technology have had limited success. In one study, glioblastoma cell lines were found to have highly heterogeneous gene expression profiles, both qualitatively and quantitatively. (Rhee, C. H. et al., cDNA expression array reveals heterogeneous gene expression profiles in three glioblastoma cell lines, Oncogene 18(17):2711-17 [1999]). In another study, a single gene was differentially overexpressed in glioblastoma multiforme tumor tissue compared to normal brain tissue. (Ljubimova, J. Y. et al., Gene expression array technique in the identification of differentially expressed genes in human brain tumors, Proceedings of the American Association for Cancer Research 40:604-05, Abstract #3986 [March 1999]). GBM tissue from two samples was shown to overexpress genes previously identified with malignancy, such as human renal cell carcinoma antigen RAGE-1, epidermal growth factor receptor, insulin-like growth factor-II, insulin-like growth factor binding protein precursors 3 and 5, fibronectin, and vimentin, compared to normal brain tissue, while expression of tubulin beta 5 and SH3-domain GRB2-like 3 was downregulated. (Ljubimova, J. Y. et al., Study of outcome prediction of patients with glial tumors by gene array comparative expression, Proceedings of the American Association for Cancer Research 41:254, Abstract #1620 [March 2000]).

It is also desirable to be able to target specific therapeutic modalities to pathogenetically distinct tumor types to maximize efficacy and minimize toxicity to the patient. (See, e.g., Golub, T. R. et al., Molecular classification of of cancer: class discovery and class prediction, Science 286:531-37 [1999]; Kudoh, K. et al., Monitoring expression profiles of doxorubicin-induced and doxorubicin-resistant cancer cells by cDNA microarray, Cancer Res. 60(15):4161-66 [2000]). Previously, cancer classification has been based primarily on the morphological appearance of tumor cells. But this has serious limitations, because tumors with similar histopathgological appearance can follow significantly different clinical courses and show different responses to therapy. For example, based on histopathological appearance, astrocytoma grade IV cannot consistently be distinguished from astrocytoma grade II.

Immunophenotyping for brain tumors has defined and refined diagnosis, e.g., distinguishing oligoastrocytoma from malignant astrocytomas, and high-grade from low-grade astrocytomas. However, differential protein expression (GFAP, vimentin, synaptophysin, nestin) has not helped to improve therapeutic approaches. Prediction of transitions from low- to high-grade astrocytomas is difficult to make with currently available markers (De Girolami, U. et al., The central nervous system. In: Cotran RC, Kumar V, Robbins S L. Pathologic basis of disease, 5th ed., pp. 1295-1357. W. B. Saunders Co. [1994]).

Tews et al. reported that immunohistochemical detection of various cancer-associated markers failed to reveal significant differential expression patterns among primary and secondary glioblastomas and precursor tumors; there was also no intraindividual constant expression pattern during glioma progression nor correlation with malignancy. (Tews, D. S. et al., Expression of adhesion factors and degrading proteins in primary and secondary glioblastomas and their precursor tumors, Invasion Metastasis 18(5-6):271-84 [1998-99]). In contrast, class prediction for leukemia has been described based on monitoring gene expression profiles with DNA microarrays. (Golub, T. R. et al. [1999]).

But no class prediction capability, based on gene expression profiles, has been available heretofore for classifying gliomas to allow for optimizing treatment regimens. Further, brain tissue adjacent to resected glioma tumors, from which secondary tumors can eventually develop, cannot be distinguished from normal brain tissue by current histopathological methods. Therefore, it is also a desideratum to be able to predict the recurrence of glioma after resection and, thus, to be able to direct aggressive treatment to sites most likely to host a recurrence These and other benefits are provided by the present invention.

### SUMMARY OF THE INVENTION

The present invention relates to a method of detecting a malignant tumor in a human subject. The method involves collecting a sample of a bodily substance containing human nucleic acid or protein originating from cells of the human subject, detecting quantitatively or semi-quantitatively in the sample a level of expression for laminin α4 subunit protein or laminin a4-specific MRNA, and comparing the expression level in the sample to a level of expression in a normal control. Overexpression of laminin α4 subunit protein or laminin α4-specific mRNA, with respect to the control, indicates the presence of a malignant tumor in the human subject. The method provides the practitioner with the ability to screen for the presence of malignant neoplasms in patients with a diagnostic test that can be done routinely and relatively cheaply to screen large numbers of people for cancerous tumors, including but not limited to, brain tumors. The method is useful both before and after clinical symptoms have appeared, and the method can also be applied to monitoring the effectiveness of anti-cancer treatments.

The present invention is based on the discovery, described herein, that malignant tumor tissues, such as glioblastoma multiforme (GBM) tissue, and particularly vascular tissue of malignant tumors, overexpress the gene encoding laminin α4 subunit, which is a constituent of the extracellular matrix protein laminin-8, compared to weak expression in normal tissue, benign tumor tissue (e.g., meningioma), and lower grade malignant tumors (e.g., astrocytoma grade II).

The present invention also relates to a method of predicting the recurrence of a malignant tumor in a human subject from whom a tumor has been resected. The method involves obtaining a tissue sample from the human subject, which tissue sample is from a region adjacent to the site of the malignant tumor. The level of expression for laminin α4 subunit protein or laminin α4-specific mRNA in the sample is detected by quantitative or semi-quantitative means, and the result is compared to the level of expression in a normal tissue control. Overexpression of laminin α4 subunit protein or laminin α4-specific mRNA, with respect to the control, is predictive of a recurrence of a malignant tumor in the subject.

In particular, the present invention also relates to a method of diagnosing the presence of a glioma in a human subject, and also provides a useful method for predicting the recurrence of a glioma in a human subject from whom a glioma has previously been resected. The method involves obtaining a tissue sample from the brain of the human subject, detecting quantitatively or semi-quantitatively a level of expression for laminin α4 subunit protein or laminin α4-specific mRNA in the sample, and comparing the expression level of laminin α4 subunit protein or laminin α4-specific mRNA in the sample to a level of expression in a normal tissue control. Overexpression of laminin α4 subunit protein or laminin α4-specific mRNA, with respect to the control, indicates the presence of glioma in the subject.

The method of predicting a recurrence of a glioma in a human subject from whom a glioma has been resected involves similar steps as described above with respect to the method of diagnosing the presence of a glioma in a human subject, except that if the tissue sample is from a brain region adjacent to the site of a glioma to be resected or adjacent to the site of a previously resected glioma, overexpression of laminin α4 subunit protein or laminin α4- specific mRNA, with respect to the control, is predictive of a recurrence of glioma in the subject. Thus, based on the molecular biological characteristics of tumor-adjacent tissue samples, which can be histopathologically normal in appearance, a relative probability of glioma recurrence after resection can be determined that enables the practitioner to adopt and monitor an appropriately modulated treatment regimen that optimizes, on an individualized basis, both therapeutic effectiveness and the quality of life for the patient.

In practicing any of the inventive methods, conventional immunochemical assay techniques can be employed to detect the expression level of laminin α4 subunit protein, or molecular biological techniques, such as but not limited to, RT-PCR and/or gene expression microarray technology (e.g., “DNA chips”), can be employed to detect the level of expression of laminin α4-specific mRNA by direct and indirect means. Gene expression microarray technology allows the construction of gene expression profiles comprising simultaneous expression levels of laminin α4-specific mRNA along with numerous other genetic sequences, such as those encoding growth factors, transcription factors, and/or structural proteins related to tumor aggressiveness and/or invasiveness.

Accordingly, the present invention is also useful as a method of classifying the grade of a malignant tumor, such as a glial tumor, in a human subject. The method involves obtaining a tissue sample from the human subject, for example from the subject's brain, which sample contains a cell expressing a plurality of mRNA species that are detectably distinct from one another, detecting quantitatively or semi-quantitatively an expression level for each of two or more of the mRNA species, one of which is a laminin α4-specific mRNA. In addition, at least one of the detected mRNA species is specific to a growth factor-related gene or to a structural gene other than a laminin gene. An expression profile of the sample is constructed, which includes a combination of the detected expression levels of laminin α4-specific mRNA and the at least one other mRNA species specific to the growth factor-related gene or to the structural gene other than a laminin gene. The expression profile is compared to an expression profile for an appropriate normal tissue control. Overexpression of laminin α4-specific mRNA, with respect to the control, indicates the presence and relatively high invasiveness of a malignant tumor in the subject Overexpression of the structural gene other than a laminin gene is indicative of relatively high tumor invasiveness, and overexpression of the growth factor-related gene is indicative of relatively high tumor aggressiveness.

Alternatively, the method of classifying the grade of a malignant tumor in a human subject, such as a glioma, is practiced with respect to detecting expression of protein gene products. A tissue sample is obtained from the human subject, which sample contains cells expressing a plurality of protein species that are detectably distinct from one another. An expression level for at least two of the plurality of protein species is detected quantitatively or semi-quantitatively. At least one of the detected protein species is a laminin α4 subunit protein and at least one is a growth factor-related protein or a structural protein other than a laminin protein. An expression profile of the sample is constructed that comprises a combination of the detected expression levels of laminin α4 subunit protein and the at least one other growth factor-related protein and/or the structural protein other than a laminin protein. The expression profile is compared to an expression profile for an appropriate normal tissue control; overexpression of laminin α4 subunit protein, with respect to the control, is indicative of the presence and relatively high invasiveness of a malignant tumor in the subject, wherein overexpression of the structural protein other than a laminin protein is indicative of relatively high tumor invasiveness. Overexpression of the growth factor-related protein is indicative of relatively high tumor aggressiveness.

The inventive method of classifying the grade of a malignant tumor, such as a glioma, in a human subject thus enables the practitioner to classify a tumor, which may be histopathologically indistinguishable from tumor in other classes, and to optimize the treatment regimen for an individual patient. Prospects for patient survival are thereby enhanced.

These and other advantages and features of the present invention will be described more fully in a detailed description of the preferred embodiments which follows.

### BRIEF DESCRIPTION OF THE DRAWINGS

FIG. 1A shows comparative gene expression by normal human brain tissue versus human corpus callosum tissue. The graph shows the overwhelming majority of gene expression levels being different only within the error range (2-fold expression difference). FIG. 1B illustrates typical comparative gene expression of glioblastoma multiforme (GBM) from Patient No. 22 versus gene expression in normal human corpus callosum tissue. FIG. 1C and 1D show typical differential gene expression in GBM tumor (FIG. 1C) or tumor-adjacent (FIG. 1D) tissues from Patient No. 39 versus corpus callosum.

FIG. 2 shows the differential expression levels of growth factor-related genes in several malignant GBMs (grade IV; columns 1-5 [in order, Patient Nos. 16, 22, 39, 45, and 50), astrocytomas grade II (Columns 6-7 [Patient Nos. 34 and 53, respectively]), and normal brain tissue (Column 8), with respect to corpus callosum internal control. Bars from left to right within each column show differential expression for the following eight genes: (i) insulin-like growth factor binding protein precursor 3; (ii) transforming growth factor; (iii) connective tissue growth factor; (iv) human insulin-like growth factor binding protein precursor 5; (v) placental growth factor; (vi) transcription factor AP-2; (vii) human insulin-like growth factor II; (viii) epidermal growth factor receptor. Most of the genes have higher expression in GBM than in the astrocytoma grade II. Therefore, for this gene group, overexpression of its members correlates with tumor aggressiveness.

FIG. 3 demonstrates the upregulation of genes encoding structural proteins in malignant brain tumors (GBMs grade IV; columns 1-5 [in order, Patient Nos. 16, 22, 39, 45, and 50), malignant brain tumors (astrocytoma grade II; columns 6-7 [Patient Nos. 34 and 53, respectively]), and normal brain tissues (column 8). Bars from left to right within each column show differential expression for the following nine genes: (i) keratin 18; (ii) vimentin; (iii) fibronectin 1; (iv) phospholipase A2 receptor; (v) desmoplakin (vi) tropomodulin; (vii) hexabrachion (tenascin C); (viii) collagen type IV α1 chain; (ix) laminin α4.

FIG. 4 demonstrates the upregulation of (FIG. 4A) “growth factor-related” genes, and (FIG. 4B) “structural” genes (including extracellular matrix protein-encoding genes) from Patient Nos. 16 and 39. Column 1 corresponds to gene expression for primary tumor from Patient No. 16(T); column 2 corresponds to tissue adjacent to primary tumor from Patient No. 16(A); Column 3 corresponds to gene expression for primary tumor from Patient No. 39(T); column 4 corresponds to tissue adjacent to primary tumor from Patient No. 39(A). Column 5 corresponds to gene expression for normal human brain tissue. In FIG. 4A, bars from left to right within each column show differential expression for the following eight genes encoding: (i) insulin-like growth factor binding protein precursor 3; (ii) connective tissue growth factor; (iii) human insulin-like growth factor II; (iv) placental growth factor; (v) transcription factor AP-2; (vi) human insulin-like growth factor binding protein precursor 5; (vii) transforming growth factor-β-induced; (viii) epidermal growth factor receptor. In FIG. 4B, bars from left to right within each column show differential expression for the following nine genes encoding: (i) keratin 18; (ii) vimentin; (iii) fibronectin 1; (iv) phospholipase A2 receptor; (v) desmoplakin (vi) tropomodulin; (vii) hexabrachion (tenascin C); (viii) collagen; (ix) laminin α4.

FIG. 5 shows the results of semiquantitative RT-PCR analysis of gene expression in brain tumors. Top, expression of the 362 bp gene of laminin α4 subunit; bottom, expression of the 333 bp fragment of β₂-microglobulin gene. Lanes are as follows: (1) GBM from Patient No. 16, primary tumor; (2) adjacent tissue to the GBM of Patient No. 16; (3) GBM of Patient No. 22; (4) GBM of Patient No. 39, primary tumor; (5) adjacent tissue to the GBM of Patient No. 39; (6) GBM of Patient No. 45; (7) GBM of Patient No. 50; (8) GBM of Patient No. 47; (9) GBM of Patient No. 25, primary tumor; (10) adjacent tissue to the GBM of Patient No. 25; (11) astrocytoma grade II of Patient No. 34; (12) meningioma (benign tumor) of Patient No. 38; (13) normal brain tissue of Patient No. 46; (14) normal brain tissue of Patient No. 40; (15) corpus callosum; (16) control without RT; (M) 100 bp DNA ladder.

FIG. 6 shows immunofluorescent staining of the distribution of laminin α4 chain-containing laminins in normal brain tissue and malignant brain tissue. Top row: N-normal brain tissue; Middle row: ACII=astrocytoma grade II; bottom row GBM (glioblastoma multiforme=astrocytoma grade IV). Left to right: immunostaining for laminin a 4, laminin β1, laminin β2, and laminin γ1 chains.

FIG. 7 shows laminin subunit expression as determined by GEM in GBM tissue samples from Patients Nos. 16T (column 1), 22 (column 2), 39 (column 3), 45 (column 4), and 50 (column 5); in astrocytoma grade II tissue samples from Patient Nos. 34 (column 6) and 53 (column 7); benign meningioma tumor (Patient No. 38, column 8); and normal brain tissue. Bars from left to right within each column show differential expression for the following seven laminin subunit genes: (i) β1; (ii) α2; (iii) α3; (iv) β3; (v) β2; (vi) γ1; and (vii) α4

### DETAILED DESCRIPTION OF THE PREFERRED EMBODIMENTS

The present invention relates to a method of diagnosing the presence of a malignant tumor in a human subject. Malignant tumors include primary, recurrent, and/or or metastatic cancerous tumors originating in any tissues, for example, carcinomas, sarcomas, lymphomas, mesotheliomas, melanomas, gliomas, nephroblastomas, glioblastomas, oligodendrogliomas, astrocytomas, ependymomas, primitive neuroectodermal tumors, atypical meningiomas, malignant meningiomas, or neuroblastomas, originating in the pituitary, hypothalamus, lung, kidney, adrenal, ureter, bladder, urethra, breast, prostate, testis, skull, brain, spine, thorax, peritoneum, ovary, uterus, stomach, liver, bowel, colon, rectum, bone, lymphatic system, skin, or in any other organ or tissue of the subject.

Thus, the present invention also relates to a method of diagnosing the presence of a glioma in a human subject. Gliomas include any malignant glial tumor, i.e., a tumor derived from a transformed glial cell. A glial cell includes a cell that has one or more glial-specific features, associated with a glial cell type, including a morphological, physiological and/or immunological feature specific to a glial cell (e.g. astrocytes or oligodendrocytes), for example, expression of the astroglial marker fibrillary acidic protein (GFAP) or the oligodendroglial marker O4. Gliomas include, but are not limited to, astrocytoma grade II, anaplastic astrocytoma grade III, astrocytoma with oligodendrogliomal component, oligodendroglioma, and glioblastoma multiforme (GBM; astrocytoma grade IV).

The inventive method involves collecting or otherwise obtaining a sample of a bodily substance derived from the human subject, which sample contains human nucleic acid or protein originating from the subject, and quantitatively or semi-quantitatively detecting therein overexpression or lack thereof of a laminin α4 gene. This includes detection by means of measuring of laminin α4 subunit proteins or laminin α4-specific nucleic acids, such as RNA or cDNA. Overexpression of laminin α4 is diagnostic for the presence of a malignant tumor or neoplasm.

The sample is preferably collected directly from the human subject's body. Preferred and convenient substances for sampling include blood, lymph or plasma, urine, cerebrospinal fluid, skin, stroma, vascular epithelium, oral epithelium, vaginal epithelium, cervical epithelium, uterine epithelium, intestinal epithelium, bronchial epithelium, esophageal epithelium, or mesothelium, or other biopsy sample of cellular material from any tissue. Cellular material includes any sample containing human cells, including samples of tissue, expressed tissue fluids (e.g., lymph or plasma), tissue wash or rinsate fluids (e.g., bladder or vaginal wash or rinsate fluids), or the like. Tissue samples that can be collected include, but are not limited to, cell-containing material from the brain, kidney, ureter, bladder, urethra, thyroid, parotid gland, submaxillary gland, sublingual gland, lymph node, bone, cartilage, lung, mediastinum, breast, uterus, ovary, testis, prostate, cervix uteri, endometrium, pancreas, liver, spleen, kidney, adrenal, esophagus, stomach, and/or intestine.

In some preferred embodiments, the sample of a bodily substance is a tissue sample from the subject's brain. This includes normal brain tissue, tumor tissue, tumor-adjacent tissue, and/or blood plasma from a site within the brain.

In accordance with the inventive methods, the tissue sample preferably contains cells that express a plurality of protein species and mRNA species, which proteins and/or mRNA species are detectably distinct from one another.

“Obtaining” and “collecting” the sample are used interchangeably herein and encompass sampling, resecting, removing from in situ, aspirating, receiving, gathering, and/or transporting the tissue sample or a concentrate, sediment, precipitate, supernatant, filtrate, aspirate, or other fraction of any of these. For example, conventional biopsy methods are useful for obtaining the tissue sample. These include percutaneous biopsy, laparoscopic biopsy, surgical resection, tissue scrapes and swabs, sampling via stents, catheters, endoscopes, needles, surgical resection, and other known means. For example, to obtain a sample from inside the skull of the human subject; typically, Magnetic Resonance Imaging (MRI)-guided stereotactic techniques are employed, but other methods can be used.

The sample is alternatively derived from cultured human cells, cell-free extracts, or other specimens indirectly derived from a subject's body, as well as from substances taken directly from a subject's body. Samples may be stored before detection methods are applied (for example nucleic acid amplification and/or analysis, or immunochemical detection) by well known storage means that will preserve nucleic acids or proteins in a detectable and/or analyzable condition, such as quick freezing, or a controlled freezing regime, in the presence of a cryoprotectant, for example, dimethyl sulfoxide (DMSO), trehalose, glycerol, or propanediol-sucrose. Samples may also be pooled before or after storage for purposes of amplifying their laminin α4 subunit-specific nucleic acids for analysis and detection, or for purposes of detecting laminin α4 subunit protein.

The sample is used immediately or optionally pre-treated by refrigerated or frozen storage overnight, by dilution, by phenol-chloroform extraction, or by other like means, to remove factors that may inhibit various amplification reactions; such as heme-containing pigments or urinary factors. For example, such amplification-inhibitory urinary factors are especially prevalent in the urine of pregnant and non-pregnant females. (E.g., J. Mahony et al., Urine specimens from pregnant and non-pregnant women inhibitory to amplification of Chlamydia trachomatis nucleic acid by PCR, ligase chain reaction, and transcription-mediated amplification: identification of urinary substances associated with inhibition and removal of inhibitory activity, J. Clin. Microbiol. 36(11):3122-26 [1998]).

The level of expression in the sample for laminin α4 subunit protein or laminin α4-specific messenger ribonucleic acid (mRNA) is then detected quantitatively or semi-quantitatively. Laminin α4 subunit protein is a polypeptide which can self-assemble with a laminin β subunit and a laminin γ subunit to form a laminin protein, which is a protein generally found in vivo as a component of the extracellular matrix. With respect to the inventive methods, useful laminin α4 subunits are found aggregated in a complete laminin protein or disaggregated therefrom, either partially (i.e., β or γ subunit is missing from the laminin protein) or fully (i.e., separated α4 subunit molecule). Laminin α4 is a constituent of laminin-8. Laminins are components of the extracellular matrix of basement membranes and are major constituents of blood vessel walls. Laminin-8 is associated with neovascularization. Thus, laminin 8 contributes to the aggressiveness and/or invasiveness of tumors. However, the present invention is not limited by nor does it depend on any particular mechanism by which expression levels of laminin-8 mediate cancer aggressiveness or invasiveness.

With respect to laminin subunits, such as laminin α4, or other proteins, the words “subunit”, “protein”, “polypeptide”, “peptide”, or “chain” are used interchangeably herein. For example, among those skilled in the art, laminin α4 subunit is commonly called “laminin α4 chain.”

Laminin α4 gene-specific polynucleotides, including laminin α4-specific mRNA species, are determined by base sequence similarity or homology to known mammalian laminin α4-specific nucleotide sequences. Base sequence homology is determined by conducting a base sequence similarity search of a genomics data base, such as the GenBank database of the National Center for Biotechnology Information (NCBI; www.ncbi.nlm.nih.gov/BLAST/), using a computerized algorithm, such as PowerBLAST, QBLAST, PSI-BLAST, PHI-BLAST, gapped or ungapped BLAST, or the “Align” program through the Baylor College of Medicine server (www.hgsc.bcm.tmc.edu/seq_data). (E.g., Altchul, S. F., et al., Gapped BLAST and PSI-BLAST: a new generation of protein database search programs, Nucleic Acids Res. 25(17):3389-402 [1997]; Zhang, J., & Madden, T. L., PowerBLAST: a new network BLAST application for interactive or automated sequence analysis and annotation, Genome Res. 7(6):649-56 [1997]; Madden, T. L., et al., Applications of network BLAST server, Methods Enzymol. 266:131-41 [1996]; Altschul, S. F., et al., Basic local alignment search tool, J. Mol. Biol. 215(3):403-10 [1990]). Preferably, a laminin α4-specific polynucleotide sequence, including an mRNA sequence, is at least 5 to 30 contiguous nucleotides long, more preferably at least 6 to 15 contiguous nucleotides long, and most preferably at least 7 to 10 contiguous nucleotides long. Preferably, the laminin α4-specific mRNA is at least about 45 contiguous nucleotides long. A laminin α4-specific mRNA can be, but is not necessarily, an mRNA species containing a nucleotide sequence that encodes a functional laminin α4 subunit or a fragment thereof. Also included among laminin α4-specific mRNAs are splice variants.

Quantitatively or semi-qunatitatively detecting the expression levels of laminin α4 subunit protein or laminin α-specific mRNAs, or of other proteins or mRNA species of interest in accordance with the present invention, is done by any known method that provides a quantitative or semi-quantitative determination of expression. A “quantitative” detection method provides an absolute value for the amount or level of expression in comparison to a standard, which amount or level is typically a mole, mass, or activity value normalized in terms of a specified mass of protein, mass of nucleic acid, number or mass of cells, body weight, or the like. Additionally, the quantitative or absolute value is optionally normalized in terms of a specified time period, i.e., expression level as a rate. A “semi-quantitative detection method provides a unitless relative value for the amount or level of expression, for example, in terms of a ratio of expression in a given sample relative to a control, such as normal tissue or the expression of a selected “housekeeping” gene. The skilled artisan is aware of other examples of quantitative and semi-quantitative detection methods.

In accordance with the inventive methods, the expression level of laminin α4 subunit protein is optionally detected by immunochemical means, such as, but not limited to, enzyme-linked immunosorbent assay (ELISA), immunofluorescent assay (IFA), immunoelectrophoresis, immunochromatographic assay or immunohistochemical staining, employing anti-lamini polyclonal or monoclonal antibodies or antibody fragments, for example Fab, Fab′, F(ab′)₂, or F(v) fragments, that selectively or specifically bind laminin α4 subunit protein. Antibodies or antibody fragments that target laminin α4 subunit are available commercially or can be produced by conventional means.

Similarly, the expression levels of other proteins of interest, in accordance with the inventive methods, can be detected by conventional immunochemical means as described above. These proteins include, but are not limited to, laminin β1 subunit, insulin-like growth factor binding protein precursor 3, transforming growth factor-β-induced gene, vascular endothelial growth factor, connective tissue growth factor, human insulin-like growth factor binding protein precursor 5, placental growth factor, transcription factor Ap-2, human insulin-like growth factor II, epidermal growth factor receptor, matrix metalloproteinase-2, keratin 18, vimentin, fibronectin 1, phospholipase A2 receptor, desmoplakin, tropomodulin, tenascin C, and collagen type IV α1 chain.

Most preferably, quantitative or semi-quantitative detection of the expression level of mRNA species is accomplished by any of numerous methods of nucleic acid amplification (e.g., amplification of laminin α4-specific nucleic acid segments) in the form of RNA or cDNA, which RNA or cDNA amplification product is ultimately measured after amplification. The final amplification product of RNA or cDNA is measured by any conventional means, such as but not limited to, densitometry, fluorescence detection, or any other suitable biochemical or physical assay system. Before amplification, it is preferable to extract or separate mRNA from genomic DNA in the sample and to amplify nucleic acids remaining in that fraction of the sample separated from the DNA, to avoid false positives that are caused by amplification of contaminating genomic DNA in the original specimen.

In accordance with the inventive method, if laminin α4 gene-specific amplification products are present, the findings are indicative of expression of laminin α4-specific mRNAs and diagnostic of the presence of a glioma in the subject. However, for interpretation of negatives (no laminin α4-specific amplification products) analysis is preferably carried out following a control amplification of nucleic acids specific for a housekeeping gene, for example, a gene encoding β-actin, phosphofructokinase (PFK), glyceraldehyde 3-phosphate dehydrogenase, or phosphoglycerate kinase. Only if expression of the housekeeping gene is detected in the sample, is the absence of laminin α4 gene expression reliably accepted. With increasing sensitivity of amplification and analysis methods employed, it becomes increasingly preferable to determine the level of laminin α4 gene expression relative to expression of a housekeeping gene. The ratio of laminin α4 expression to housekeeping gene expression is determined, for example, by real-time PCR methods or densitometric measurement and analysis of electrophoretic bands after amplification. When the ratio of laminin α4 expression to housekeeping gene expression exceeds a normal cell standard range and/or approximates an abnormal (e.g., GBM) cell standard range, this indicates overexpression of laminin α4 gene product and is indicative of GBM or predictive of its recurrence.

The mRNAs are amplified by a suitable amplification method. For example, in a preferred embodiment, a reverse transcriptase-mediated polymerase chain reaction (RT-PCR) is employed to amplify laminin α4-specific nucleic acids. Briefly, two enzymes are used in the amplification process, a reverse transcriptase to transcribe laminin α4-specific cDNA from a laminin α4-specific mRNA template in the sample, a thermal resistant DNA polymerase (e.g., Taq polymerase), and laminin α4-specific primers to amplify the cDNA to produce laminin gene-specific amplification products. Examples of useful laminin α4-specific primers include (1) forward primer: 5′ CTCCATCTCACTGGATAATGGTACTG 3′ (SEQ. ID. NO.:1); and (2) reverse primer: 5′ GACACTCATAAAGAGAAGTGTGGACC 3′ (SEQ. ID. NO.:2). The use of limited cycle PCR yields semi-quantitative results. (E.g., Gelfand et al., Reverse transcription with thermostable DNA polymerase-high tempreature reverse transcription, U.S. Pat. Nos. 5,310,652; 5,322,770; Gelfand et al., Unconventional nucleotide substitution in temperature selective RT-PCR, U.S. Pat. No. 5,618,703).

In another preferred embodiment of the inventive method, single enzyme RT-PCR is employed to amplify laminin α4 gene-specific nucleic acids. Single enzymes now exist to perform both reverse transcription and polymerase functions, in a single reaction. For example, the Perkin Elmer recombinant Thermus thermophilus (rTth) enzyme(Roche Molecular), or other similar enzymes, are commercially available.

In another preferred embodiment, real-time RT-PCR is employed to amplify laminin α4 gene-specific nucleic acids. Briefly, this is a quantitative gene analysis based on the ratio of laminin α4 gene expression and the expression of a housekeeping gene, i.e., a gene that is expressed at about the same level in normal and abnormal (e.g., malignant) cells, for example, a gene encoding β₂-microglobulin (β₂-MG), β-actin, phosphofructokinase, glyceraldehyde 3-phosphate dehydrogenase, or phosphoglyceratekinase. The the ratio of the laminin α4 and housekeeping genes' expressions is routinely established as a standard for normal and abnormal cells, which standard expression ratio(s) is (are) used for comparison in determining that expression of the laminin α4 gene relative to expression of the “housekeeping” gene in a given sample is either “normal” or “increased”, the latter indicative of “overexpression” and diagnostic for the presence of a glioma in the subject . In this embodiment, the ratio is the key to diagnosis and constitutes quantitative gene expression analysis. This embodiment utilizes so-called real-time quantitative PCR, carried out with commercially available instruments, such as the Perkin Elmer ABI Prism 7700, the so-called Light Cycler (Roche Molecular), and/or other similar instruments. Optionally, single enzyme RT-PCR technology, for example, employing rTth enzyme, can be used in a real-time PCR system. Preferably, amplification and analysis are carried out in an automated fashion, with automated extraction of mRNA from a urine sediment sample, followed by real-time PCR, and fluorescence detection of amplification products using probes, such as TaqMan or Molecular Beacon probes. Typically, the instrumentation includes software that provides quantitative analytical results during or directly following PCR without further amplification or analytical steps.

In another preferred embodiment, transcription-mediated amplification (TMA) is employed to amplify laminin α4 gene-specific nucleic acids. (E.g., K. Kamisango et al., Quantitative detection of hepatitis B virus by transcription-mediated amplification and hybridization protection assay, J. Clin. Microbiol. 37(2):310-14 [1999]; M. Hirose et al., New method to measure telomerase activity by transcription-mediated amplification and hybridization protection assay, Clin. Chem. 44(12)2446-52 [1998]). Rather than employing RT-PCR for the amplification of a cDNA, TMA uses a probe that recognizes a laminin α4-specific (target sequence) mRNA; in subsequent steps, from a promoter sequence built into the probe, an RNA polymerase repetitively transcribes a cDNA intermediate, in effect amplifying the original mRNA transcripts and any new copies created, for a level of sensitivity approaching that of RT-PCR. The reaction takes place isothermally (one temperature), rather than cycling through different temperatures as in PCR.

Other useful amplification methods include a reverse transcriptase-mediated ligase chain reaction (RT-LCR), which has utility similar to RT-PCR. RT-LCR relies on reverse transcriptase to generate cDNA from mRNA, then DNA ligase to join adjacent synthetic oligonucleotides after they have bound the target cDNA.

Most preferably, amplification of a laminin α4 gene-specific nucleic acid segment in the sample obtained from the subject can be achieved using laminin α4 or other gene-specific oligonucleotide primers and primer sets, which are commercially available or which are synthesized by conventional methods based on known genetic sequences (e.g., see GenBank accession numbers in Tables 2-5 in Example 2 herein). Typically, a gene-specific primer is a gene-specific oligonucleotide at least 15 to 30 contiguous nucleotides long, and most preferably 17 to 22 nucleotides long, but primers as short as 7 contiguous nucleotides may be useful for some gene-specific sequences. (E.g., Vincent, J., et al., Oligonucleonucleotides as short as 7-mers can be used for PCR amplification, DNA Cell Biol. 13(1):75-82 [1994]). The skilled artisan can readily determine other useful gene-specific nucleotide sequences for use as primers or probes by conducting a sequence similarity search of a genomics data base, such as the GenBank database of the National Center for Biotechnology Information (NCBI), using a computerized algorithm, such as PowerBLAST, QBLAST, PSI-BLAST, PHI-BLAST, gapped or ungapped BLAST, or the “Align” program through the Baylor College of Medicine server, as described hereinabove.

Optionally, high throughput analysis may be achieved by PCR multiplexing techniques well known in the art, employing multiple primer sets, for example primers directed not only to laminin α4 gene-specific nucleic acids, but to amplifying expression products of housekeeping genes (controls) or of other potential diagnostic markers known in the art, e.g., oncogenes, such as MAG or telomerase, to yield additional diagnostic information. (E.g., Z. Lin et al., Multiplex genotype determination at a large number of gene loci, Proc. Natl. Acad. Sci. USA 93(6):2582-87 [1996]; Demetriou et al., Method and probe for detection of gene associated with liver neoplastic disease, U.S. Pat. No. 5,866,329).

Most preferably, gene expression microarray (“GEM”; commonly known as cDNA microarray”, “DNA chip”, or “gene chip”) analysis is employed to detect the expression level of laminin α4 mRNA and expression levels of other mRNA species of interest in accordance with the inventive methods, for example, gene-specific mRNAs encoding proteins such as laminin β1 subunit, insulin-like growth factor binding protein precursor 3, transforming growth factor-β-induced gene, vascular endothelial growth factor, connective tissue growth factor, human insulin-like growth factor binding protein precursor 5, placental growth factor, transcription factor Ap-2, human insulin-like growth factor II, epidermal growth factor receptor, matrix metalloproteinase-2, keratin 18, vimentin, fibronectin 1, phospholipase A2 receptor, desmoplakin, tropomodulin, tenascin C, and collagen type IV α1 chain. Gene expression microarrays are constructed by known methods by which a multiplicity of specific oligonucleotide sequences are attached to a solid support, such as a slide or “chip”, where PCR amplification and/or hybridization reactions are conducted in situ. (E.g., Carulli, J. P. et al., High throughput analysis of differential gene expression, J. Cell Biochem. Suppl. 30-31:286-96 [1998]; Scherer, S., Quantitative methods, systems and apparatuses for gene expression analysis, WO9958720A1; Gerhold, D. et al., DNA chips: promising toys have become powerful tools, Trends Biochem. Sci. 24(5):168-73 [1999]; Duggan, D. J. et al, Expression profiling using cDNA microarrays, Nat. Genet. 21(1 Suppl): 10-14 [1999]; Erlander, M. G. et al., Method for generating gene expression profiles, WO028092A1; Nelson, P.S. et al., Comprehensive analysies of prostate gene expression: convergence of expressed sequence tag databases, transcript profiling and proteomics, Electrophoresis 21(9): 1823-31 [2000]; De Benedetti, V. M. et al., DNA chips: the future of biomarkers, Int. J. Biol. Markers 15(1):1-9 [2000]; Bradley, A. et al., Chemically modified nucleic acids and methods for coupling nucleic acids to solid support, U.S. Pat. No. 6,048,695; Lockhart, D. J. et al., Expression monitoring by hybridization to high density oligonucleotide arrays, U.S. Pat. No. 6,040,138, Dehlinger, P. J, Position-addressable polynucleotide arrays, U. S. Pat. No. 5,723,320; Pinkel, D. et al., Comparative fluorescence hybridization to nucleic acid arrays, U.S. Pat. No. 5,830,645).

Alternatively, gene expression microarrays are employed that are available commercially, for example, by Incyte Genomics (Incyte Pharmaceuticals, Inc., Palo Alto, Calif.) or Genome Systems (St. Louis, Mo.). A gene expression profile including the expression levels of one or several of the genes of interest in accordance with the inventive methods, in any combination, can be constructed relatively easily by GEM analysis with appropriate analytical computer software, typically available from or provided by the microarray manufacturer (e.g., Incyte Genomics' GEM Tools software). However, other useful analytical methods are known to the skilled artisan for detecting differential gene expression, such as serial analysis of gene expression (SAGE), subtractive cloning, differential display, and the like. (E.g., Kinzler, K. W. et al., Method for serial analysis of gene expression, U.S. Pat. No. 5,866,330; Larsson, M. et al, Expression profile viewer (ExProView): software tool for transcriptome analysis, Genomics 63(3):341-53 [2000]; Angelastro, J. M. et al., Improved NlaIII digestion of PAGE-purified 102 bp ditags by addition of a single purification step in both SAGE, and micro SAGE protocols, Nucleic Acid Res. 28(12):E62 [2000]; Streicher, J. et al., Computer-based three-dimensional visualization of developmental gene expression, Nat. Gen. 25(2):147-52 [2000]).

Hybridization analysis is a preferred method employed in measuring or analyzing amplification products or of detecting the expression level of laminin α4-specific mRNA in total RNA isolated directly from the sample without employing an amplification. Hybridization analysis employs one or more laminin α4 gene-specific probe(s) that, under suitable conditions of stringency, hybridize(s) with single stranded laminin α4 gene-specific nucleic acid amplification products comprising complementary nucleotide sequences. The amplification products or RNA are typically deposited on a substrate, such as a cellulose or nitrocellulose membrane, and then hybridized with labeled laminin α4 gene-specific probe(s), optionally after an electrophoresis. Alternatively, hybridization reactions can be conducted using a cDNA microarray as described above, with the probe sequences attached to the microarray slide or chip. Of course, hybridization techniques can also be used to probe for mRNA species specific to genes encoding any of the other proteins of interest in accordance with the inventive methods as described above. A useful probe is typically 7 to 500 nucleotides long, most preferably 15 to 150 nucleotides long, and comprises a gene-specific nucleotide sequence, for at least part of its length. Conventional dot blot, Southern, Northern, or fluorescence in situ (FISH) hybridization protocols, in liquid hybridization, hybridization protection assays, or other semi-quantitative or quantitative hybridization analysis methods are usefully employed along with laminin α4 gene-specific probes or other gene-specific probes of interest.

The phrase “stringent hybridization” is used herein to refer to conditions under which annealed hybrids, or at least partially annealed hybrids, of polynucleic acids or other polynucleotides are stable. As known to those of skill in the art, the stability of hybrids is reflected in the melting temperature (Tₘ) of the hybrids. In general, the stability of a hybrid is a function of sodium ion concentration and temperature. Typically, the hybridization reaction is performed under conditions of relatively low stringency, followed by washes of varying, but higher, stringency. Reference to hybridization stringency relates to such washing conditions.

As used herein, the phrase “moderately stringent hybridization” refers to conditions that permit target-DNA to bind a complementary nucleic acid that has about 60% sequence identity or homology, preferably about 75% identity, more preferably about 85% identity to the target DNA; with greater than about 90% identity to target-DNA being especially preferred. Preferably, moderately stringent conditions are conditions equivalent to hybridization in 50% formamide, 5×Denhart's solution, 5×SSPE, 0.2% SDS at 42° C., followed by washing in 0.2×SSPE, 0.2% SDS, at 65° C.

The phrase “high stringency hybridization” typically refers to conditions that permit hybridization of only those nucleic acid sequences that form stable hybrids in 0.018 M NaCl at 65° C (i.e., if a hybrid is not stable in 0.018 M NaCl at 65° C, it will not be stable under high stringency conditions, as contemplated herein). High stringency conditions can be provided, for example, by hybridization in 50% formamide, 5×Denhart's solution, 5×SSPE, 0.2% SDS at 42° C., followed by washing in 0.1×SSPE, and 0.1% SDS at 65° C.

The phrase “low stringency hybridization” typically refers to conditions equivalent to hybridization in 10% formamide, 5×Denhart's solution, 6×SSPE, 0.2% SDS at 42° C., followed by washing in 1×SSPE, 0.2% SDS, at 50° C. Denhart's solution and SSPE (see, e.g., Sambrook et al., Molecular Cloning, A Laboratory Manual, Cold Spring Harbor Laboratory Press [1989]) are well known to those of skill in the art as are other suitable hybridization buffers.

Alternatively, electrophoresis for analyzing amplification products is done rapidly and with high sensitivity by using any of various methods of conventional slab or capillary electrophoresis, with which the practitioner can optionally choose to employ any facilitating means of nucleic acid fragment detection, including, but not limited to, radionuclides, UV-absorbance or laser-induced fluorescence. (K. Kepamik et al., Fast detection of a (CA)18 microsatellite repeat in the IgE receptor gene by capillary electrophoresis with laser-induced fluorescence detection, Electrophoresis 19(2);249-55 [1998]; H. Inoue et al, Enhanced separation of DNA sequencing products by capillary electrophoresis using a stepwise gradient of electric field strength, J. Chromatogr. A. 802(1):179-84 [1998]; N. J. Dovichi, DNA sequencing by capillary electrophoresis, Electrophoresis 18(12-13):2393-99 [1997]; H. Arakawa et al, Analysis of single-strand conformation polymorphisms by capillary electrophoresis with laser induced fluorescence detection, J. Pharm. Biomed. Anal. 15(9-10):1537-44 [1997]; Y. Baba, Analysis of disease-causing genes and DNA-based drugs by capillary electrophoresis. Towards DNA diagnosis and gene therapy for human diseases, J. Chromatgr B. Biomed. Appl. 687(2):271-302 [1996]; K. C. Chan et al., High-speed electrophoretic separation of DNA fragments using a short capillary, J. Chromatogr B. Biomed. Sci. Appl. 695(1):13-15 [1997]).

Any of diverse fluorescent dyes can optionally be used to label probes or primers or amplification products for ease of analysis, including but not limited to, Cy3, Cy5, SYBR Green I, YIO-PRO-1, thiazole orange, Hex (i.e., 6-carboxy-2′,4′,7′,4,7-hexachlorofluoroscein), pico green, edans, fluorescein, FAM (i.e., 6-carboxyfluorescein), or TET (i.e., 4,7,2′,7′-tetrachloro-6-carboxyfluoroscein). (E.g., J. Skeidsvoll and P. M. Ueland, Analysis of double-stranded DNA by capillary electrophoresis with laser-induced fluorescence detection using the monomeric dye SYBR green I, Anal. Biochem. 231(20):359-65 [1995]; H. Iwahana et al., Multiple fluorescence-based PCR-SSCP analysis using internal fluorescent labeling of PCR products, Biotechniques 21(30:510-14, 516-19 [1996]). Conventional fluorescence detection means can be employed to detect and measure fluorescent label quantitatively or semi-quantitatively, including flow-activated cell sorting (FACS) technology.

In accordance with the inventive method of diagnosing the presence of a malignant tumor, such as a glioma, in a human subject, the expression level detected is compared with the level of expression in an appropriate normal control. A suitable normal control is preferably a preselected sample or pooled samples of a bodily substance analogous to the particular bodily substance comprising the sample, and subjected to the same test or detection procedures as the tested sample. For example, normal kidney tissue for a kidney tissue sample, normal plasma or serum for plasma sample, normal lung tissue for a lung tissue sample, and the like. For brain tissue, as described herein, the normal tissue control can be a preselected control of corpus callosum tissue, corpus callosum tissue extract, or corpus callosum RNA, as appropriate to the techniques of detection employed, which is prepared from pooled non-pathological human corpus callosum samples or which is commercially available (e.g., Clontech, Palo Alto, Calif.) Alternatively, non-pathological human brain tissue (e.g., white and/or grey matter) samples can be pooled and are useful as a control. Also useful, but less preferred for practical, clinical and ethical reasons, is the use of a tumor-contralateral normal tissue sample from the individual human subject to be tested. However, tumor-adjacent tissue from the same individual subject is not acceptable as a normal control in accordance with the present invention, because as described herein, tumor-adjacent tissue can have an abnormal molecular expression profile, despite a histopathologically normal appearance.

Overexpression of laminin α4 subunit protein or laminin α4-specific mRNA, with respect to the control, indicates the presence of a malignant tumor, such as a glioma, in the subject. For purposes of the present invention “overexpression” means a level of expression of a protein or mRNA species, including but not limited to laminin α4 subunit protein or laminin α4-specific mRNA, at least about twice the level of expression found in the normal control, as determined quantitatively or semi-quantitatively.

A useful, but not an essential, positive control, in the event that laminin α4 subunit or laminin α4-specific mRNA overexpression is detected, is also detecting the overexpression of laminin β1 subunit protein or laminin β1-specific mRNA, which together with the laminin α4 overexpression is confirmatory for laminin-8 overexpression.

The present invention also relates to a method of predicting the recurrence of a malignant tumor, for example a glioma, in a human subject from whom a malignant tumor has been resected. The method involves obtaining a tissue sample from a region of the tissue of interest, such as the brain, of the human subject that is adjacent to the site of the tumor, which has been resected or will be resected. A tumor-adjacent region of the brain extends from immediately beyond the edge of the tumor, and up to about 2 cm beyond the edge of the tumor in situ, or beyond the former location of the tumor's edge after resection. The tumor is marked by morphologically malignant cells that are histopathologically distinct from the non-malignant cells in the tumor-adjacent region. The tumor-adjacent tissue sample is typically histopathologically normal in appearance, but it can also be hyperplastic, cytologically dysplastic and/or premalignant, or otherwise histopathologically abnormal.

Detecting quantitatively or semi-quantitatively a level of expression for laminin α4 subunit protein or laminin α4-specific mRNA in the sample is accomplished as described above. Comparing the expression level to a predetermined level of expression in a normal tissue control is also accomplished as described above. Overexpression of laminin α4 subunit protein or laminin α4-specific mRNA, with respect to the control, is predictive of a recurrence of a malignant tumor in the subject, likely developing from within the tumor-adjacent tissue that was sampled and tested in accordance with the inventive method. This does not mean that the probability of a recurrence of tumor is 1.0, but rather that the probability of tumor recurrence is greater than zero and greater than it would be that a tumor will develop at another histopatholgically normal tissue site.

Some preferred embodiments of the inventive methods include detecting quantitatively or semi-quantitatively in the sample a level of expression with respect to a normal control, of a growth factor-related gene. The “growth factor-related gene” encodes a protein involved in growth regulation, such as, but not limited to, insulin-like growth factor binding protein precursor 3, transforming growth factor-β-induced gene, vascular endothelial growth factor, connective tissue growth factor, human insulin-like growth factor binding protein precursor 5, placental growth factor, transcription factor Ap-2, human insulin-like growth factor II, and/or epidermal growth factor receptor. Overexpression of any or all of these genes is diagnostic or predictive of the relative aggressiveness of the tumor, i.e., the rapidity of neoplastic cellular proliferation.

Additionally or alternatively, some preferred embodiments of the inventive methods include detecting quantitatively or semi-quantitatively in the sample a level of expression with respect to a normal control, of a “structural” gene, i.e., a gene encoding a protein related to the extracellular matrix. Such proteins include, but are not limited to, matrix metalloproteinase-2, keratin 18, vimentin, fibronectin 1, phospholipase A2 receptor, desmoplakin, tropomodulin, tenascin C, and/or collagen type IV α1 chain. Overexpression of any or all of these genes is diagnostic or predictive of the relative invasiveness of the glioma, i.e., its ability to penetrate, encroach upon, enter, or impinge on surrounding non-malignant brain tissues.

Accordingly, the present invention relates to a method of classifying the grade of a malignant tumor in a human subject. The method involves obtaining a tissue sample, e.g., a brain tissue sample, from the human subject, as described hereinabove. Also as described above are methods for detecting quantitatively or semi-quantitatively an expression level for at least two of the plurality of detectably distinct protein species and/or mRNA species that are contained in the cells of the tissue. The method is practiced either by detecting the level of expression with respect to protein gene product and/or with respect to mRNA. At least one of the detected protein species and/or mRNA species is a laminin α4 subunit or a laminin α4-specific mRNA, respectively. At least one is a product of a growth factor-related gene or of a structural gene, as described herein. It is preferable, but not an essential feature of the method, to include in the expression profile expression levels of one or more protein species and/or mRNA species from both categories, i.e., structural and growth factor-related genes. However, at least one of the structural genes is other than a laminin gene (i.e., a gene that encodes a laminin subunit; e.g., see subunits in Table 6 in Example 2 herein).

Constructing an expression profile of the sample means assembling the expression level data resulting from the detection step into a tabular, graphical, or otherwise analytically useful combination of the detected expression levels of the protein or mRNA gene products.

Comparing the expression profile to an expression profile for a normal tissue control is diagnostically and prognostically useful. While the overexpression of laminin α4 subunit and/or laminin α4-specific mRNA, with respect to the control, indicates of the presence of a tumor, such as a glioma, the supplemental information provided by the expression profile yields more particular intelligence as to the relative aggressiveness and/or invasiveness of the glioma tumor. As stated above, overexpression of the “structural” gene other than a laminin gene is indicative of relatively high tumor invasiveness, and overexpression of the “growth factor-related” gene is indicative of relatively high tumor aggressiveness. Laminin 8 can contribute to both aggressiveness and invasiveness through its roles in the angiogenesis of neovasculature and in the extracellular matrix.

Histopathological means of classifying malignant tumors into grades are known for various kinds of malignant tumor, including gliomas. (E.g., R. C. Cotran, V. Kumar, and S. L. Robbins (eds). Pathologic basis of disease. 5th ed., pp. 1295-1357. W. B. Saunders Co.,[1994]; Kleihues, P. et al., The WHO classification of brain tumors, Brain Pathol. 3:255-268 [1993]; Daumas-Duport C. et al., Grading of gliomas: a simple and reproducible method, Cancer 62: 2152-2165 [1988]). Generally, malignant tumor tissues are classified into about four grades by experienced histopathologists, ranging from tissues containing cells of normal to slightly dysplastic appearance (grade I) to those tissues with the most severely malignant appearance (grade IV). In accordance with the method, laminin α4 subunit or laminin α4-specific mRNA are about 2.0- to about 3.5-fold overexpressed in grade II tumors, about 3.4- to about 3.8-fold overexpressed in grade III tumors, and greater than about 3.8-fold overexpressed in grade IV tumors. This applies, for example, to glial tumors of astrocytoma grades II-IV.

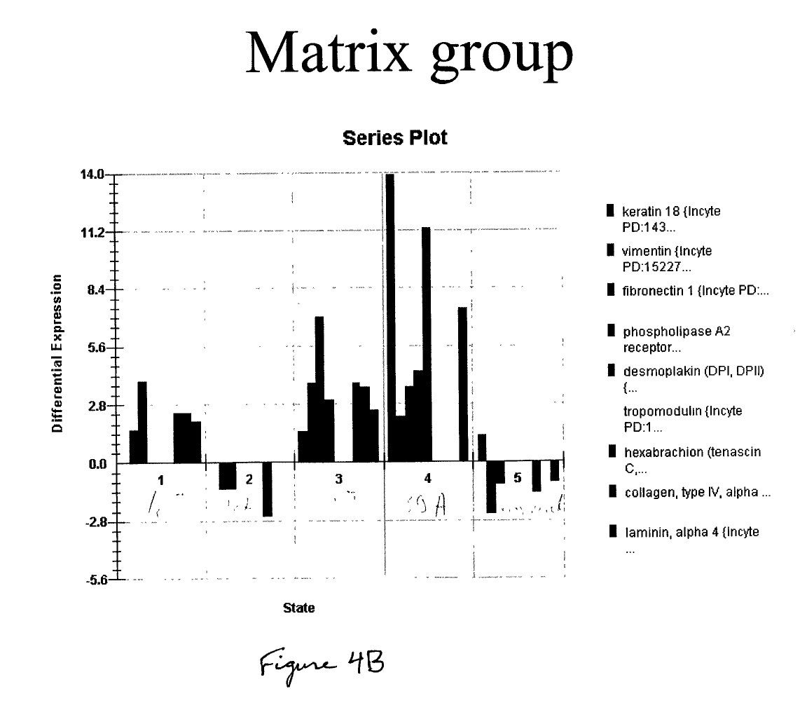

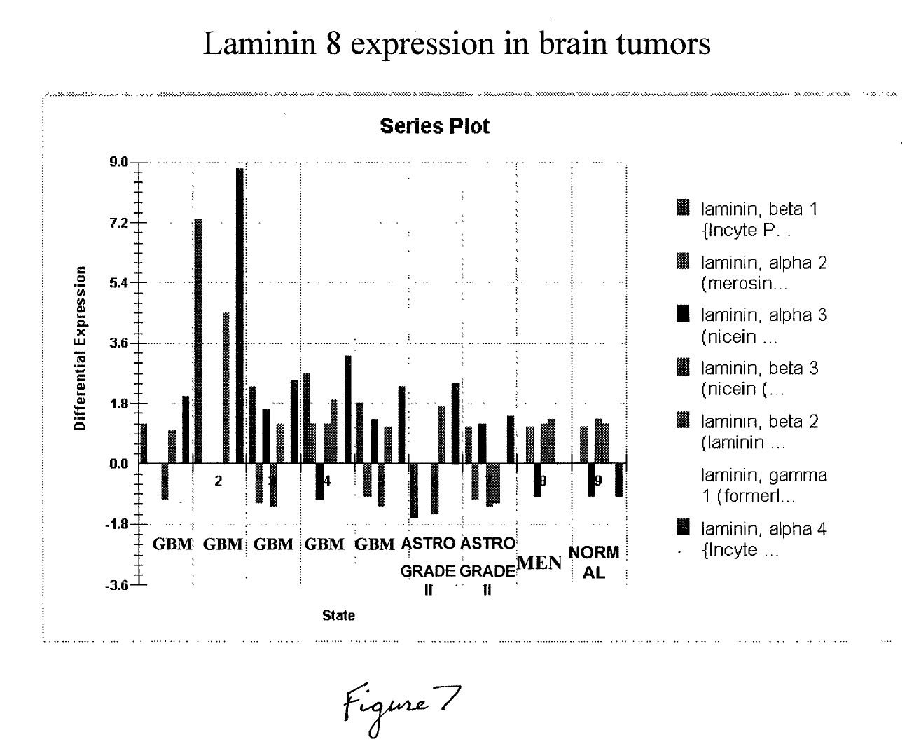

Thus the inventive method allows the practitioner to gain knowledge as to the grade of the tumor, based on its molecular expression phenotype as detected by its expression profile, which information was unavailable heretofore from histopathological observation alone. For example, GBM and astrocytoma grade II are frequently indistinguishable with conventional histopathological methods, but using the inventive method, these glioma grades are readily distinguished, since GBM generally overexpresses laminin α4 and a number of growth factor-related genes and structural genes that astrocytoma grade II typically does not. (See Tables 2 and 3 and FIGS. 5-7).

Also, even among GBMs it is possible by using the method to distinguish at least two different groups of GBMs, which were heretofore indistinguishable by conventional histopathological means. The first group contains the more aggressive GBMs (“grade IV(a)”), as exemplified by Patient No. 22, as described herein; the second group contains the less aggressive GBMs (“grade IV(b)”), as exemplified by Patient No. 16, described herein. (See also FIGS. 5-7).

The foregoing description of the methods of the present invention are illustrative and by no means exhaustive. When these features of the present invention are employed, diagnostic and treatment decisions can be more appropriately optimized for the individual glioma patient, and the prospects for his or her survival can be enhanced.

The invention will now be described in greater detail by reference to the following non-limiting examples.

### EXAMPLES

### Example 1

### Materials and Methods

Gene Expression Microarray.

A sequence-verified cDNA microarray, i.e., gene expression microarray (GEM), was introduced for the analysis of gene expression patterns for 11,004 unique human genes on a single array (6,794 gene clusters and 4,210 annotated genes) and 400 annotated ESTs (UniGEM™ V, Genome Systems, St. Louis, Mo.). Each gene sequence was about 500-5000 base pairs in length. The array required not more then 600 ng of poly(A)+RNA per experiment. Detection in the UniGem™ system was by fluorescence-based signal detection, which is safer and more sensitive than radioactivity-based detection, and included new computer software developed for array analysis, with improved GenBank links and comparative and statistical capabilities.

Microarray (GEM) Preparation.

Nucleic acid sequences used for microarray fabrication were generated by polymerase chain reaction (PCR). PCR products were purified by gel filtration with Sephacryl-400 (Amersham Pharmacia Biotech, Inc., Piscataway, N.J.) equilibrated in 0.2×SSC. The filtrate is dried down and rehydrated in one-tenth-volume dH2O for arraying. The DNA solutions are arrayed by robotics on modified glass slides. After arraying, slides are processed to fix the DNA to the prepared glass surface and washed three times in dH₂O at room temperature. Slides are then treated with 0.2% I-Block (Tropix, Bedford, Mass.), dissolved in 1×Dulbecco's phosphate PCR products were purified by gel filtration with Sephacryl-400 (Amersham Pharmacia Biotech, Inc., Piscataway, N.J.), equilibrated in 0.2×SSC. The filtrate was dried down and rehydrated in one-tenth-volume dH₂O for application to the gene expression array. The DNA solutions were arrayed by robotics on modified glass slides (microarray slides).

After DNA was applied to the microarray slides, the slides were processed to fix the DNA to the prepared glass surface and were washed three times in dH₂O at room temperature. Microarray slides were then treated with 0.2% I-Block (Tropix, Bedford, Mass.), which was dissolved in 1×Dulbecco's phosphate buffered saline (Life Technologies, Gaithersburg, Md.) at 60° C. for 30 minutes. The GEM microarrays were then rinsed in 0.2% SDS for two minutes, followed by three one-minute washes in dH₂O.

Tissue samples.

Fresh human glioma samples were obtained from the Department of Pathology and Laboratory Medicine, Cedars-Sinai Medical Center, and were frozen in liquid nitrogen and stored at −80° C. immediately after surgery until RNA extraction and morphological evaluation and tumor grading. A total of 27 tissue samples were used for gene expression microarray analysis, RT-PCR and immunohistochemistry, including 15 primary gliomas, 5 adjacent tissues to the gliomas from the same patients, 3 meningiomas (benign brain tumors), 3 normal brain tissues from trauma patients and one corpus callosum. (See, Table 1).

TABLE 1
Tissue samples used for GEM analysis.

| | | | | Number of |
|---|---|---|---|---|
| Patient | | | | genes regulated |
| code | Age/Sex | Tissue type | Tumor grade | up/down |
| 16T* | 45/M | GBM | IV | 256/183 |
| 16A* | 45/M | Adjacent to GBM | normal | 330/180 |
| 22 | 58/M | GBM | IV | 593/332 |
| 39T** | 38/M | GBM | IV | 806/37 |
| 39A** | 38/M | Adjacent to GBM | normal | 186/148 |
| 45 | 70/M | GBM | IV | 111/108 |
| 50 | 61/M | GBM | IV | 249/59 |
| 34 | 48/M | Astrocytoma | II | 121/176 |
| 53 | 32/M | Astrocytoma | II | 79/49 |
| 38 | 46/M | Meningioma | Benign tumor | 36/74 |
| 46 | 38/F | Trauma patient | normal | 81/86 |
| 44 | 47/M | Trauma patient | normal | 45/67 |

Samples marked with * (i.e., 16T, 16A) and ** (i.e., 39T and 39A) each designate one patient with primary tumor and corresponding adjacent tissue.

Tumor grading was based on the WHO classification and Daumas-Duport et al (Kleihues, P. et al., The WHO classification of brain tumors, Brain Pathol. 3:255-268 [1993]; Daumas-Duport C. et al., Grading of gliomas: a simple and reproducible method, Cancer 62: 2152-2165 [1988]). All 12 experimental tissues in Table 1 were compared to poly (A)+RNA from human corpus callosum (pool from 70 tissues donors), which was used as an internal control, because corpus callosum consists mainly of glial cell types (Lue, L. F. et al., Characterization of glial cultures from rapid autopsies of Alzheimer's and control patients, Neurobiol. Aging 17:421-429 [1996]), and therefore, seems to be an adequate normal control for glial tumors.

For gene expression microarray analysis, 12 experimental tissues were used including five GBMs, two brain tissues adjacent to two of five GBMs, two astrocytomas grade II, one meningioma and two normal brains from trauma patients. All these samples were compared to normal human corpus callosum used as an internal control tissue. The Poly(A)+RNAs were obtained from normal adult human corpus callosum (pooled mRNAs obtained from 70 trauma patients), purchased from Clontech (Palo Alto, Calif.). The gene expression profiles of two histologically normal adjacent tissue samples (from the same patient with two of five GBMs) was also evaluated against normal corpus callosum (see, Table 1, footnote). In accordance with the manufacturer's (Clontech) protocol, all balanced differential expression ratios higher than 2 were considered significant.

Fluorescent Labeling of Probe.

Poly(A)+RNA (mnRNA) was isolated from tissue samples as described previously (Ljubimova, J. Y. et al., Novel human malignancy associated gene (MAG) expressed in various tumors and in some tumor preexisting conditions, Cancer Res. 58:4475-79 [1998]). Isolated mRNA was reverse-transcribed with 5′ Cy3- or Cy5-labeled random 9-mers (Operon Technologies, Inc., Alameda, Calif.). Cy3 was used to label probes for hybridization with RNA samples from corpus callosum {internal controls); Cy5 was used to label probes for hybridization with RNA from tumor tissue samples.

Reactions were incubated for 2 hours at 37° C. with 200 ng poly(A) RNA, 200 Units M-MLV reverse transcriptase (Life Technologies, Gaithersburg, Md.), 4 mM DTT, 1 unit RNase Inhibitor (Ambion, Austin, Tex.), 0.5 mM dNTPs, and 2 mg of labeled 9-mers in a 25-mL volume with enzyme buffer supplied by the manufacturer. The reactions was terminated by incubation at 85° C. for 5 min. The paired reaction mixtures were combined and then purified with a TE-30 column (Clontech, Palo Alto, Calif.), brought to 90-μL volume with dH₂O, which was precipitated with 2 μL of 1 mg/mL glycogen, 60 μL 5M ammonium acetate, and 300 μL ethanol. After centrifugation, the supernatant was decanted and the pellet was resuspended in 24 μL of hybridization buffer: 5×SSC, 0.2% SDS, 1 mM DTT.

Hybridization.

Probe solutions were thoroughly resuspended by incubating at 65° C. for 5 minutes with mixing. The probe was applied to the microarray, which was then covered with a 22 mm² glass cover-slip and was placed in a sealed chamber to prevent evaporation. After hybridization at 60° C. for 6.5 hours, slides were washed in three consecutive washes of decreasing ionic strength.

Scanning.

Microarrays were scanned in both Cy3 and Cy5 channels with Axon GenePix scanners (Axon Instruments, Inc., Foster City, Calif.) with a 10 μm resolution. The signal was converted into 16-bits-per-pixel resolution, yielding a 65,536 count dynamic range.

Normalization and Ratio Determination.

Incyte Genomics' GEM Tools computer software (Incyte Pharmaceuticals, Inc., Palo Alto, Calif.) was used for image analysis. The elements were determined by a gridding and region detection algorithm. The area surrounding each element image was used to calculate a local background and was subtracted from the total element signal. Background subtracted element signals were used to calculate Cy3 (expression in corpus callosum control):Cy5 (expression in tumor tissue) ratios. The average of the resulting total Cy3 and Cy5 signal gave a ratio that was used to balance or normalize the signals.

Semiquantitative Reverse Transcription-polymerase Chain Reaction (RT-PCR).

This was carried out essentially as described previously (Ljubimova, J. Y. et al., Expression of HGF, its receptor (c-met), c-myc, and albumin in cirrhotic and neoplastic human liver tissue, J. Histochem. Cytochem. 45:79-87 [1997]). cDNA was synthesized from 2.0 μg total RNA in 80 μL of reaction buffer containing 500 μM dNTPs, 2.5 ptM random hexamer primers, 20 U RNase inhibitor and 200 U SuperScript II reverse transcriptase (Life Technologies). The reaction was first carried out for 10 min. at 25° C., then for 30 min. at 42° C., followed by 5 min. at 95° C. and subsequent cooling to 4° C. cDNA samples were subjected to PCR using specific primers for gene array-selected laminin α4 chain gene and for β₂-microglobulin (β₂-MG) gene that served as a standard for sample normalization. Primers listed below were designed using Primer3 Internet software program (The Whitehead Institute, Boston, Mass.) and their specificity was confirmed by BLAST Internet software-assisted search (Altschul, S. F. et al., Gapped BLAST and PSI-BLAST: a new generation of protein database search programs, Nucleic Acids Res. 25:3389-3402 [1997]) of a non-redundant nucleotide sequence database (National Library of Medicine, Bethesda, Md).

The following primers were used to amplify laminin α4-specific nucleic acid:

| | |
|---|---|
| (1) forward primer: | |
| 5′ CTCCATCTCACTGGATAATGGTACTG 3′ | (SEQ. ID. NO.: 1), |
| (2) reverse primer: | |
| 5′ GACACTCATAAAGAGAAGTGTGGACC 3′ | (SEQ. ID. NO.: 2) |

The following primers were used to amplify β₂-MG-specific nucleic acid:

| | |
|---|---|
| (1) forward primer: | |
| 5′ CTCGCGCTACTCTCTCTTTCTG 3′ | (SEQ. ID. NO.: 3); |
| (2) reverse primer: | |
| 5′ GCTTACATGTCTCGATCCCACTT 3′ | (SEQ. ID. NO.: 4). |

PCR was carried out with 100 ng of reverse-transcribed poly(A)+RNA (in some cases, total RNA was used), Taq polymerase buffer (Promega) containing 200 μM dNTPs, 1.25 U Taq polymerase and 250 nM of sense and anti-sense primers, in a total volume of 50 μL. Each cycle consisted of 30 sec. denaturation at 94° C., 30 sec. annealing, and 45 sec. elongation at 72° C, and 35 cycles were performed for laminin α4-specific nucleic acid. Amplification of β₂-MG-specific nucleic acid was used to normalize the samples. Normalized samples were amplified in a linear range established using serial cDNA dilutions and varying the number of cycles. Negative controls without reverse transcriptase and water control, and a positive kit control were included in each reaction. Amplified products were electrophoretically separated in 3% agarose gels, visualized and photographed under UV light after ethidium bromide staining.

To confirm the specificity of PCR products, selected bands were excised from the gels, purified using Wizard PCR Prep (Promega), reamplified, cloned into Plasmid PCR II using a TA cloning kit (Invitrogen, Carlsbad, Calif.), and sequenced in an automatic DNA sequencer 373 (Applied Biosystems, Foster City, Calif.).

Immunofluorescent Analysis.

19 tissue samples were used: 12 primary glial tumors, three adjacent tissues to GBMs, two meningiomas, and two normal brains There were nine GBMs and three astrocytomas grade II-III among the glial tumors. Nine tissue samples belonged to the same cases where laminin α4 chain gene expression was also analyzed by gene expression microarray and semiquantitative RT-PCR. Tissue samples were snap-frozen in liquid nitrogen by a pathologist immediately after surgery and then embedded in OCT compound for cryosectioning. Cryo stat sections of 8 μm thickness were processed for determination of indirect immunofluorescence as described previously (Ljubimov, A. V. et al., Human corneal basement membrane heterogeneity: topographical differences in the expression of type IV collagen and laminin isoforms, Lab. Invest. 72:461-73 [1995]; Ljubimov, A. V. et al., Human corneal epithelial basement membrane and integrin alterations in diabetes and diabetic retinopathy, J. Histochem. Cytochem. 46:1033-41 [1998]). Well-characterized polyclonal and monoclonal antibodies against laminin subunits α1, α2 (clone 1F9), α3 (BM165), α4, β1 (clone LT3), β2 (clone C4), β3 (clone 6F12), and γ1 (clones A5 and 2E8) were used as previously described (Ljubimov et al. [1995]; Ljubimov et al. [1998]; Sorokin et al. [2000]; Miner et al. [1997]). The monoclonal antibody to laminin α5 chain (clone 4C7) as well as secondary cross-species absorbed fluorescein- and rhodamine-conjugated donkey anti-mouse, anti-rat and anti-rabbit antibodies were from Chemicon International. Two different polyclonal antibodies to laminin α4 chain gave very similar results. The same was true for two monoclonal antibodies to laminin γ1 chain. Routine specificity controls (without primary or secondary antibodies) were negative. Monoclonal antibodies were used as straight hybridoma supernatants or at 10-20 μg/mL when purified, and polyclonal antibodies were used at 20-30 μg/mL. At least two independent experiments were performed for each marker, with identical results. Sections were viewed and photographed with an Olympus BH-2 fluorescence microscope.

Statistical Analysis.

Statistical analysis was done using the two-sided Fisher's exact test.

### Example 2

### Results

Reliability of Corpus Callosum Internal Control.

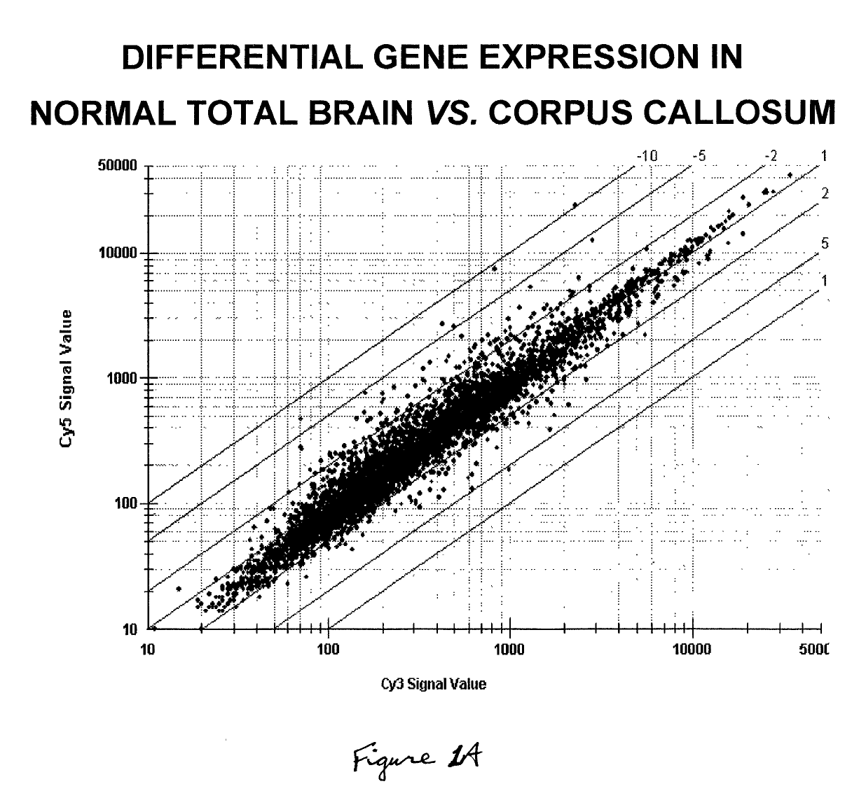

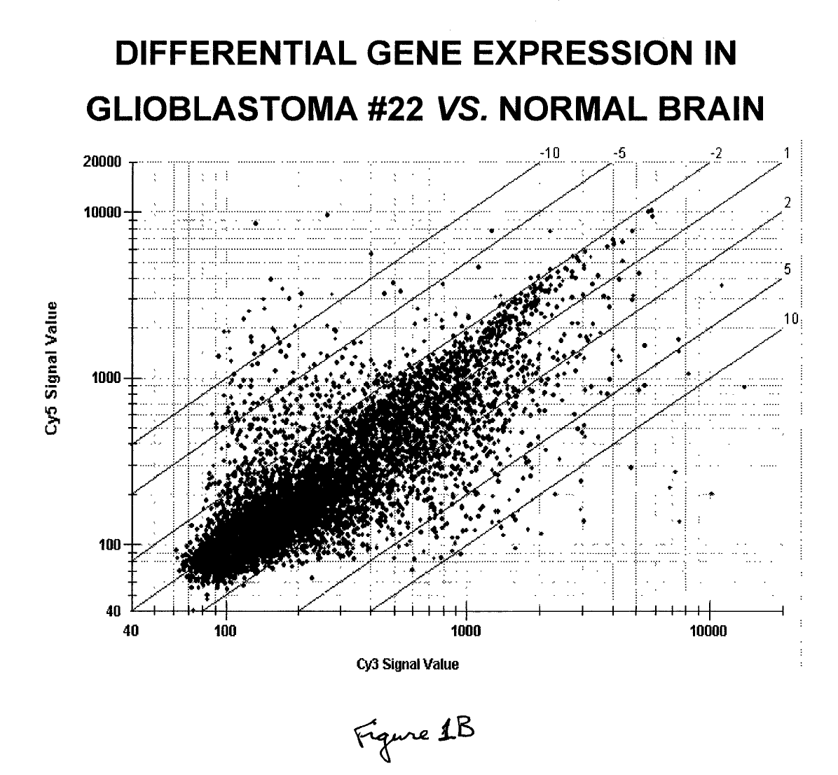

The effectiveness of selecting corpus callosum tissue as the normal internal control for brain tissue was confirmed by comparing gene expression in corpus callosum with normal brain tissue (mostly white matter). Importantly, when normal brain tissue was compared to corpus callosum tissue, no major differences in gene expression were found (FIG. 1A). In FIG. 1A, the overwhelming majority of gene expression levels in normal brain tissue differed from gene expression in corpus callosum tissue only within the error range, i.e., within the 2× difference as defined by the manufacturer. The expression of some genes, such as ectodermal-neural cortex protein (ratio of 4.8) and synapsin II (ratio of 3.2) may reflect the differenses in normal metabolic processes rather than significant transformation changes that occur in the process of malignancy. In sum, FIG. 1A shows that normal brain tissue and corpus callo sum tissue have about the same gene expression profile.

Differential Gene Expression in Malignant Tumor Tissues.

Gene expression in glioblastoma multiformes (GBM) tissue differed significantly from expression in control tissue. A typical example is shown in FIG. 1B, where gene expression in GBM tissue from Patient No. 22 was compared to control. In contrast, gene expression in GBM-adjacent tissue was more similar to gene expression in GBM than to gene expression in normal brain tissue, as shown for example by a comparision of FIG. 1C [Patient No. 39T] with FIG. 1D [Patient No. 39A] and FIG. 1A.

Gene expression analysis of 5 primary GBMs by GEM microarray detected a total of 2345 genes with increased expression and 719 genes with decreased expression compared to corpus callosum. Of these genes, 14 genes were significantly upregulated in all 5 GBMs (Tables 2 and 3) and 12 genes were downregulated in all 5 GBMs (Table 5). The majority of downregulated genes play a role in metabolic processes (Table 5). Among the overexpressed genes (Tables 2 and 3), some genes have been previously associated with gliomas, and other genes were never described in gliomas before.

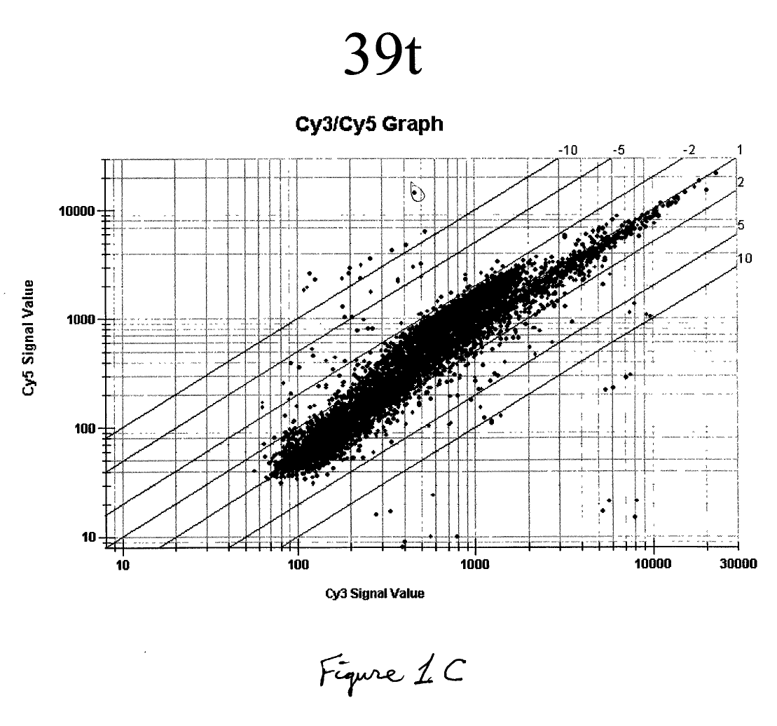

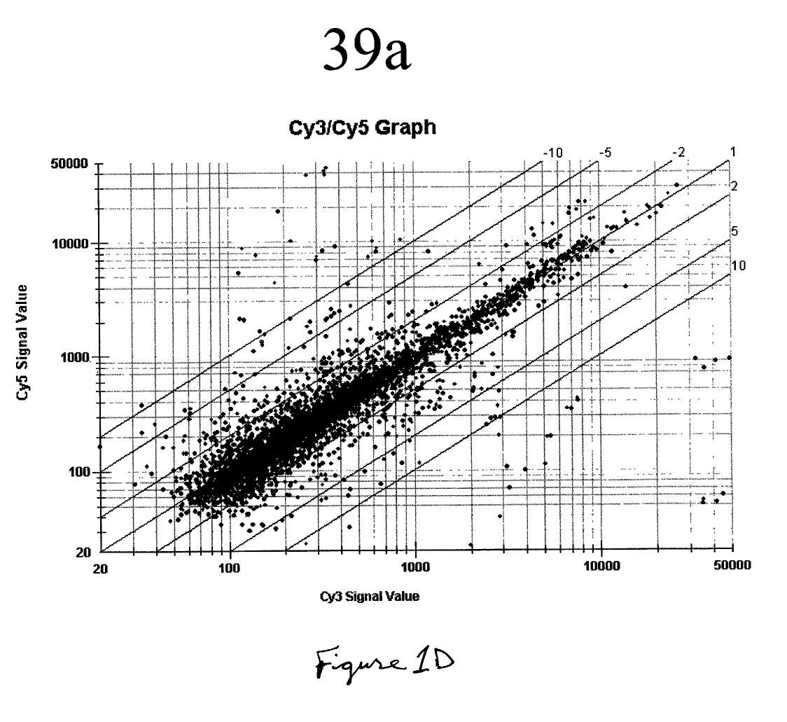

The genes that were overexpressed in GBMs could be arbitrarily divided into two groups. The first gene group coded for proteins related to the growth process, such as transcription factor AP-2, EGF receptor, IGF binding protein precursor 3, IGF binding protein precursor 5, IGF-II, TGF-β-induced gene, VEGF, and connective tissue growth factor. Average ratios of expression of these genes in gliomas compared to normal brain tissue are shown in Table 2. Elevated expression of all these genes apparently reflects the active growth process in GBMs. (See FIG. 2).

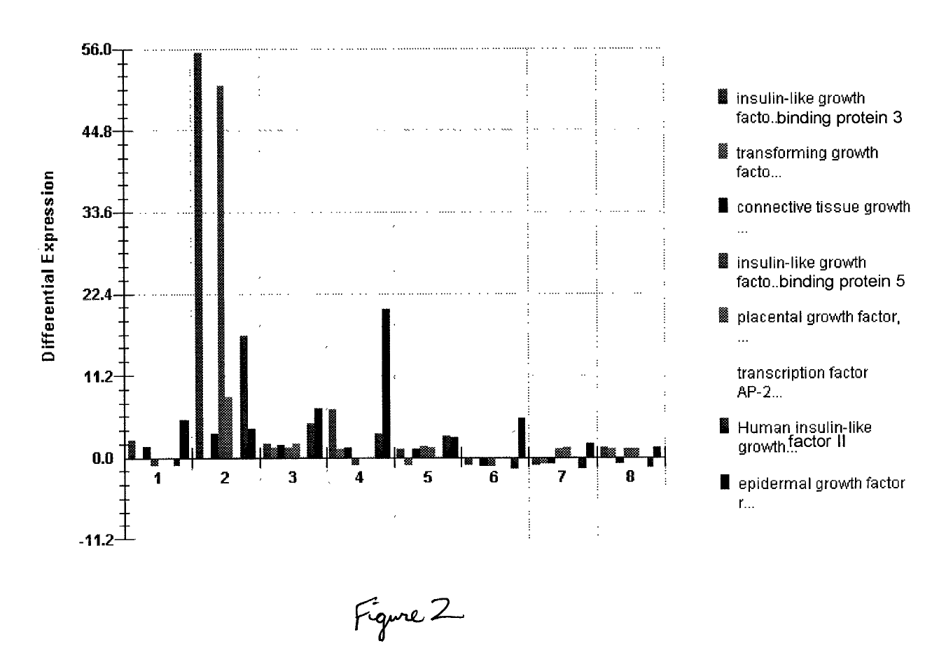

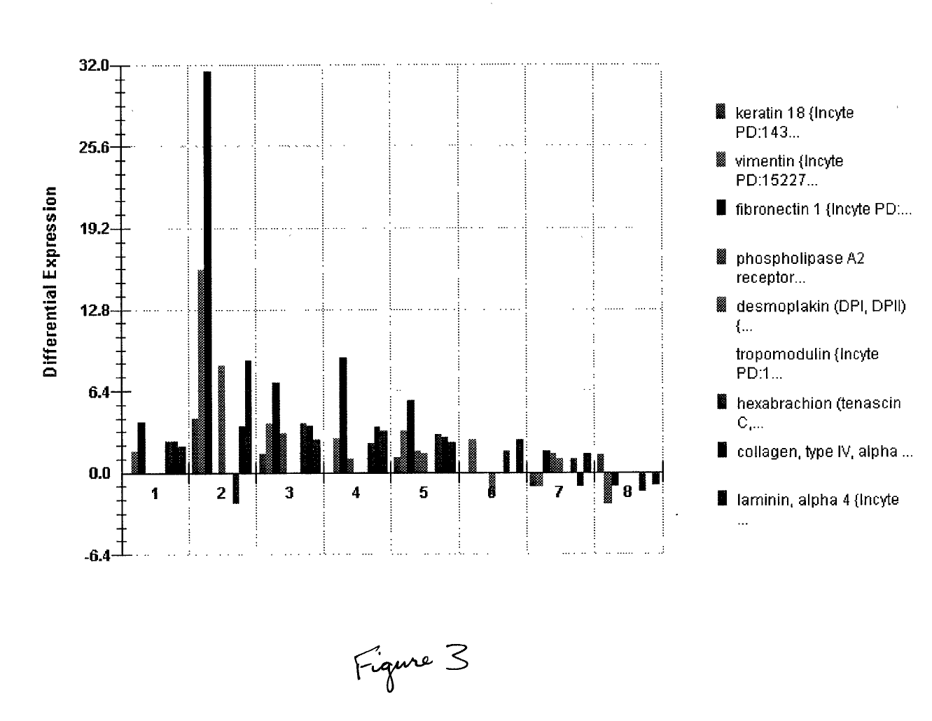

The second group is represented by genes coding for structural proteins, including ECM-related proteins, such as vimentin, fibronectin, tenascin-C, type IV collagen α1 chain, phospholipase A2 receptor, laminin α4 chain, keratin 18, desmoplakin and tropomodulin. Table 3 and FIG. 3 show expression levels of 10 genes encoding structural proteins and extracellular matrix proteins (i.e., “structural” genes) that are overexpressed in all human gliomas studied. Most of these structural genes had higher expression in GBM (grade IV) than in the astrocytoma (grade II). Most of the genes have higher expression in GBM than in the astrocytoma. Interestingly, some of these genes were also overexpressed in GBM-adjacent tissue, but not in astrocytoma grade II. Therefore, for this gene group, overexpression of its members correlates with tumor aggressiveness. Overexpression of laminin α4 subunit isoform, has not previously been identified with any known tumor (Table 3).

Expression of several selected genes was common to all glial tumors examined (Table 1), including five grade IV glioblastomas and two grade II astrocytomas. Gene expression profiles of tumor-adjacent tissue more closely resembled the expression profiles of GBMs (grade IV) than the expression profiles of grade II tumors (FIGS. 2 and 3). The GEM analysis identified upregulation and downregulation of genes that are differentially expressed in malignant brain tumors. There were about 57 genes that were up regulated in all gliomas examined (i.e., grade IV and grade II glial tumors) and 115 genes that were down regulated.

In all glial tumors examined, there were 20 overexpressed genes that are known to code for growth factors and structural proteins. Using the gene expression microarray method, quantitative comparison of the expression of these 20 genes in tumors was made at different stages of tumor progression (Tables 2 and 3). The mean ratio for growth factor-related gene expression in GBMs (grade IV) was higher than in astrocytomas grade II.

The results of the GEM analysis showed that in the five GBMs there was an overexpression of certain growth factor-related genes that were previously associated with tumor growth and invasion. In particular, Table 2 and FIG. 2 present expression levels of growth factor-related genes in GBM tissues. Average ratios of expression compared to normal brain tissue are shown. For each of the five glioblastomas listed in Table 1, all genes that are related to growth factors were overexpressed compared to normal brain tissue control (corpus callosum control). Elevated expression of all these genes reflects the active growth process in GBMs.

After detection of significant differential expression of 14 genes in five GBMs compared to normal brain tissue, it was determine how these genes were expressed in other glial tumors with lower grade. Two astrocytomas grade II from Patient Nos. 34 and 53, one benign meningioma from Patient No. 38, and two normal brain tissues from Patient Nos. 44 and 46 were studied (Table 1 and FIGS. 2 and 3; gene profile for Patient Nos. 38 and 44 not shown).

Using GEM array and corresponding image analysis software, we simultaneously compared 14 genes of interest in each tumor in a quantitative manner (FIGS. 2 and 3). The mean expression ratio for growth factor-related genes in GBMs (grade IV) was higher than in astrocytomas grade II. The gene expression microarray analysis in astrocytomas grade II from Patient Nos. 34 and 50 identified only two growth-related genes that were significantly overexpressed, transcription factor AP-2 and EGF receptor (Table 2). From the structural (including ECM)-related gene group, only laminin α4 subunit gene was significantly overexpressed in both low-grade astrocytoma and GBM compared to normal brain (Table 3; FIGS. 2 and 3).

TABLE 2
Expression of growth factor-related genes

| | GBM | Adjacent | Astrocytoma | GeneBank |
|---|---|---|---|---|
| Gene | Grade IV | to GBM | Grade II | Accession # |
| Transcription factor AP-2 | 8.3 | 1.2 | 2.1 | M36711 |
| Epidermal growth factor | 7.8 | 2.4 | 3.6 | X00588 |
| receptor | | | | |
| Insulin-like growth factor | 13.4 | 1.8 | 0.0 | M31159 |
| binding protein precursor 3 | | | | |
| Human insulin-like growth | 5.3 | 0.6 | 0.0 | L27560 |
| factor binding protein precursor 5 | | | | |
| Insulin-like growth factor II | 10.3 | 6.6 | 0.0 | AW411300 |
| Transforming growth factor- | 6.7 | 2.7 | 0.5 | AC004503 |
| β-induced gene | | | | |
| Vascular endothelial growth factor | 2.3 | 2.4 | 0.7 | AI004656 |
| Connective tissue growth factor | 1.8 | 3.6 | 0.0 | U14750 |

Overexpression of growth-related genes such as transcription factor AP-2, EGF receptor, IGF-II, and VEGF was described previously in glial tumors, most often one gene at a time (Tysnes et al. [1999]; Mäenp\ddot{aa} et al. [1997]; Kilic et al. [2000]; Qin et al. [1999]; Melino et al. [1992]). When gene expression in human gliomas was examined using the gene expression microarray method, coordinate overexpression of these genes was observed in all gliomas studied. In addition, some other genes, such as IGF binding protein precursor 3, IGF binding protein precursor 5, and laminin α4 subunit chain, were shown for the first time to be overexpressed in gliomas compared to normal brain tissue (Table 2).

The gene expression microarray analysis described herein demonstrates significant upregulation of 14 genes in five GBMs. Respective patients were followed up and it is very interesting that the clinical course agreed well with gene profiles of primary GBMs and two corresponding adjacent tissues from the same patients. For example, Patient Nos. 22, 45, and 39 had overexpression of genes for transcription factor AP-2, EGF receptor, IGF binding protein precursor 3, and IGF-II that are known to promote tumor growth. The corresponding tumors revealed higher expression of these genes than GBMs from Patient Nos. 16 and 50. Structural (including ECM) protein encoding genes such as vimentin, fibronectin, and laminin-8 were also highly expressed in Patient Nos. 22, 39, and 45. The tumor gene profiles of Patients Nos. 22, 45, 39 could thus be regarded as “more aggressive/malignant” compared to the tumor gene profile of Patient Nos. 16 and 50. Notably, the former three patients developed recurrences every 2-3 months and had 2-3 surgeries before they died. However, Patient No. 50 did not develop a recurrence for more than 9 months up to at least the time this application was filed.

TABLE 3
Expression of structural genes.

| | GBM | Adjacent | Astrocytoma | GeneBank |
|---|---|---|---|---|
| Gene | Grade IV | to GBM | Grade II | Accession # |
| Matrix | 3.1 | 1.4 | 0.0 | J03210 |
| metalloproteinase-2 | | | | |
| Vimentin | 5.4 | 0.5 | 0.8 | X56134 |
| Fibronectin | 11.4 | 1.2 | 0.8 | AW385690 |
| Tenascin-C | 2.0 | 0.0 | 1.4 | NM_002160 |
| Collagen type IV | 3.2 | 0.0 | 0.6 | M26576 |
| α1 chain | | | | |
| Phospholipase | 1.1 | 2.2 | 0.8 | U17033 |
| A2 receptor | | | | |
| Laminin α4 chain | 3.8 | 3.7 | 2.0 | Z99289 |
| Keratin 18 | 1.4 | 7.0 | 0.6 | NM_000224 |
| Desmoplakin | 2.0 | 5.7 | 0.4 | J05211 |
| Tropomodulin | 2.4 | 2.4 | 1.6 | NM_003275 |

Average ratios of expression compared to normal brain tissue are shown

Tumor-adjacent Tissues.

A feature of tumor-adjacent tissues was that they had high expression levels of 10 genes out of 14 that were overexpressed in GBMs (Table 2; Table 3). In cases of glioblastoma multiforme, the main sites from which develop recurrence of tumors are these histologically normal adjacent tissues. For growth factor-related genes and structural proteins that are involved in the process of invasion the differential gene expression was even more pronounced between glial tumors of different grades and adjacent histologically normal-looking tissues.

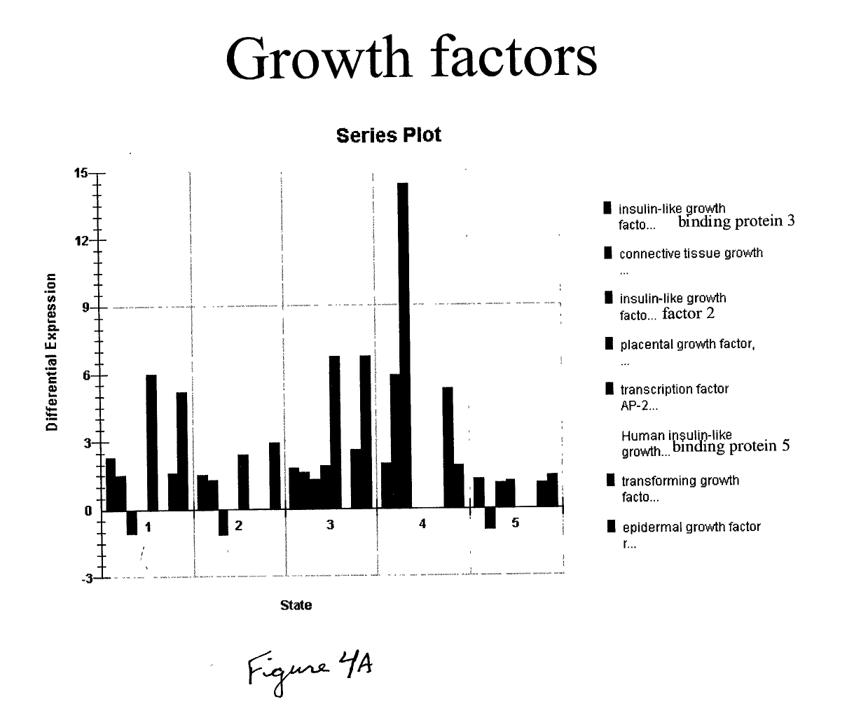

Two histologically normal tumor-adjacent tissues (judged histologically normal by a board-certified pathologist) were compared with corresponding primary tumors from Patient Nos. 16 and 39 (FIG. 4A and FIG. 4B, compare column 1 with column 2 and column 3 with column 4). The gene array analysis yielded different gene profiles for tumor-adjacent tissue from each patient. Gene profiles of both adjacent tissues were more similar to GBMs than to normal tissue despite their normal histology. Some genes that were upregulated in a number of glial tumors, such as connective tissue growth factor, VEGF, keratin 18, desmoplakin, and laminin α4 subunit, had higher or similar expression levels in histologically normal tumor-adjacent tissues compared to primary GBM (Tables 2 and 3). In particular, tumor-adjacent tissue exhibited higher expression of keratin 18, desmoplakin, phospholipase A2 receptor, and connective tissue growth factor, compared to GBM tissue.

It is worth noting that the gene expression levels for tumor-adjacent tissues for Patient Nos. 16A (FIGS. 4A and 4B, column 2) and 39A (FIGS. 4A and 4B, column 4), while being intermediate compared to expression for these genes in GBM and low grade tumors, where also different from each other. GBM from Patient No. 16 and its adjacent tissue had on average lower levels of most GBM-related genes compared to GBM from Patient No. 39 and its adjacent tissue. The more intense overexpression of genes from Patient No. 39 compared to Patient No.16, in both primary tumor and adjacent histologically normal tissue correlated with the more dismal clinical prognosis of Patient No. 39, who developed new tumor recurrences approximately every two months after tumor resections, in comparison with Patient No. 16, who developed tumor recurrences more slowly and survived 12 month longer than Patient No. 39. This observation was consistent with the comparative clinical courses for seven selected patients with GBM (Patient Nos. 16, 22, 39, 42, 45, 49, and 50). All seven were followed up, and their clinical course was comparable with gene profiles of primary GBMs and adjacent tissues.

Being more intensively expressed in histologically normal tumor-adjacent tissues than in primary GBM, some of the overexpressed genes, for example, epithelial markers keratin 18 and desmoplakin, connective tissue growth factor, phospholipase A2 receptor, and laminin α4 subunit, may play a significant role in tumor development and progression. The expression of some tumor-related genes in adjacent tissues before the appearance of morphological changes means that some of these genes probably play a role in tumor development and progression. The fact of gene expression, similar to GBM, in histologically normal tissue adjacent to GBM, confirms the hypothesis that tumor invasion is a process of significant molecular changes that happen before phenotypic and morphologic alterations are detectable. Despite a general normal histological appearance, these tissues may have had microinvasive foci that could have contributed to tumor-like gene expression pattern. An alternative explanation may be that tumor-derived factors could increase expression of specific genes in tumor-adjacent tissues, such as keratin 18, desmoplakin, connective tissue growth factor, phospholipase A2 receptor, and laminin α4 subunit.

Low-grade tumors exhibited markedly less overexpression than GBM and tumor-adjacent tissues. The gene expression ratio for the of the astrocytomas grades II were significantly lower than in GBMs (Table 4 and Table 5 below). The genes overexpressed are presumably genes that regulate the growth of low-grade tumors and may promote the tumor progression. These genes, such as transcription factors 8 and 12, have relatively low expression levels in GBMs that overexpressed transcription factor II.

For ESTs, with unknown genetic identity and function, there were similar levels of expression in primery tumors compared to adjacent tissues. Some ESTs were overexpressed in low grade tumors (astrocytoma grade TI), but not in GBM tissue. (Table 4 below). General analysis of gene expression in astrocytomas grade II (without genes selected for five GBMs) demonstrated overexpression of 17 known genes and two ESTs in astrocytomas grade II in comparison with corpus callosum (Table 4).

TABLE 4
Gene expression in astrocytoma grade II tumors compared to normal control
(corpus callosum; N) for genetic sequences that overexpressed in low grade (LG)
but not in glioblastoma multiforme.

| | Expression Ratio | Accession |
|---|---|---|
| Genetic Sequence Name | grade II/N | No. |
| transcription factor AP-2 | 2.1 | M36711 |
| transcription factor 12, HTF4 | 3.8 | M65209 |
| transcription factor 8 | 2.9 | U19969 |
| aldehyde dehydrogenase 9 | 3.1 | U50203 |
| apolipoprotein E | 6.4 | NM_000041 |
| chondroitin sulfate proteoglycan 2 (versican) | 4.7 | X15998 |
| dihydropyrimidinase-like 3 | 4.6 | NM001387 |
| EST | 5.6 | AI686533 |
| EST | 4.2 | AL133916 |
| glutamate dehydrogenase 1 | 3.5 | X07769 |
| human BAC clone RG118D07 from 7q31 | 4.4 | AC004142 |
| hypoxia-inducible factor 1 α subunit | 2.5 | U29165 |
| (basic helix-loop-helix transcription factor) | | |
| microtubule-associated protein 2 | 4.0 | U89329 |
| myristoylated alanine-rich protein kinase C substrate | 2.9 | D10522 |
| neurotrophic tyrosine kinase receptor, type 2 | 5.7 | NM006180 |
| nuclear factor I/B | 4.1 | NM005596 |
| peripheral myelin protein 2 | 3.1 | D16181 |
| poly(A)-binding protein, cytoplasmic 1 | 2.5 | Y00345 |
| protein tyrosine phosphatase, receptor-type, zeta | 5.5 | NM002851 |
| polypeptide 1 | | |
| ribosomal protein L6 | 2.4 | AI888138 |
| transcription factor 12, HTF4 | 3.8 | M65209 |
| transcription factor 8 (represses interleukin 2 expression) | 2.9 | U19969 |

Gene profile analysis revealed that there are only two genes: that are down regulated in two astrocytomas grade II (not shown).

Gene expression microarray analysis also showed a number of downregulated genes, as well as upregulated genes. Table 5 lists some of the genes downregulated in glioblastoma compared to normal brain tissue.

TABLE 5
Down-regulation of gene expression in glioblastoma multiforme (GBM)
compared to normal tissue (corpus callosum; N).

| | Expression Ratio | Accession |
|---|---|---|
| Genetic Sequence Name | GBM/N | No. |
| calpain, large polypeptide L3 | −6.9 | AI147217 |
| cerebellar degeneration-related protein (34 kD) | −25.6 | NM004065 |
| DKFZP586D0823 protein | −6.0 | AI761105 |
| EGF-like repeats and discoidin I-like domains 3 | −9.2 | NM005711 |
| EST | −5.8 | AA782011 |
| Homo sapiens clone 23664 and 23905 mRNA sequence | −5.6 | AF035315 |
| myelin-associated glycoprotein | −19.2 | X98405 |
| myelin-associated oligodendrocyte basic protein | −35.0 | H23197 |
| peanut (Drosophila)-like 2 | −13.2 | AI632238 |
| protease, serine, 9 (neurosin) | −14.5 | D78203 |
| proteolipid protein 1 (Pelizaeus-Merzbacher disease, | −69.8 | M27110 |
| spastic paraplegia2) | | |
| S100 calcium-binding protein β(neural) | −16.5 | NM006272 |

Semiquantitative RT-PCR.

Since laminin α4 subunit gene upregulation was typical for GBMs, GBM adjacent tissues, and low-grade astrocytomas, its expression was analyzed further by semiquantitative RT-PCR to confirm the gene expression microarray data. Semiquantitative RT-PCR was used to study seven primary GBMs, histologically verified GBM-adjacent tissues from three patients, one astrocytoma grade II, one meningioma, two normal brain tissues from trauma patients and one sample from corpus callosum. All GBMs that had been analyzed by gene microarray were included in semiquantitative RT-PCR. The results confirmed the gene array analysis data. All GBMs and their adjacent tissues highly expressed laminin α4 subunit gene. Meningioma from patient 38 and normal brain from patient 46 had lower levels of laminin α4 subunit gene expression than glial tumors, but higher than normal brain from Patient No. 44 and corpus callosum (FIG. 5).

Immunohistochemical Study.

Generally, laminins are components of basement membranes and the major constituents of blood vessel walls. Therefore, in malignant tumors, including brain tumors such as gliomas, laminin can be associated with neovascularization and contribute to the aggressiveness and/or invasiveness of tumors. Table 6 shows the constituent subunit polypeptide chains of 12 of the known isoforms of laminin.

TABLE 6
Known isoforms of laminin (From Miner, 1999.
Kidney Int. 56:2016-2024)

| Isoform | heterotrimer |
|---|---|
| Laminin-1 | α1β1γ1 |
| Laminin-2 | α2β1γ1 |
| Laminin-3 | α1β2γ1 |
| Laminin-4 | α2β2γ1 |
| Laminin-5 | α3β3γ2 |
| Laminin-6 | α3β1γ1 |
| Laminin-7 | α3β2γ1 |
| Laminin-8 | α4β1γ1 |
| Laminin-9 | α4β2γ1 |
| Laminin-10 | α5β1γ1 |
| Laminin-11 | α5β2γ1 |
| Laminin-12 | α2β1γ3 |

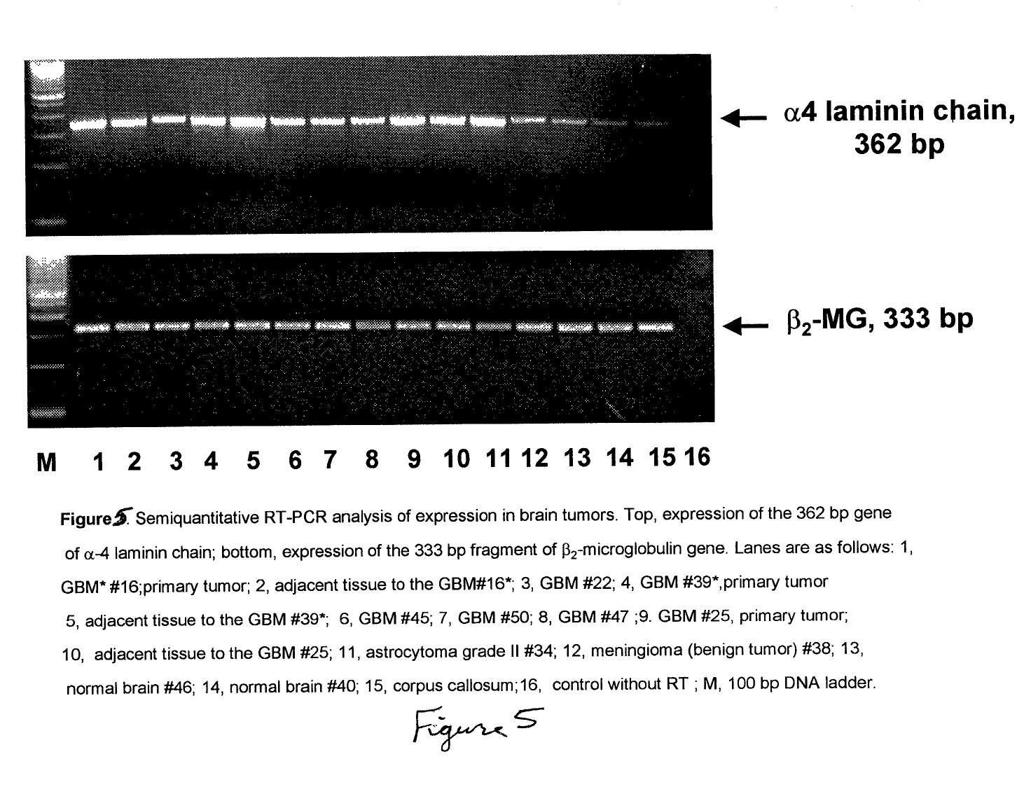

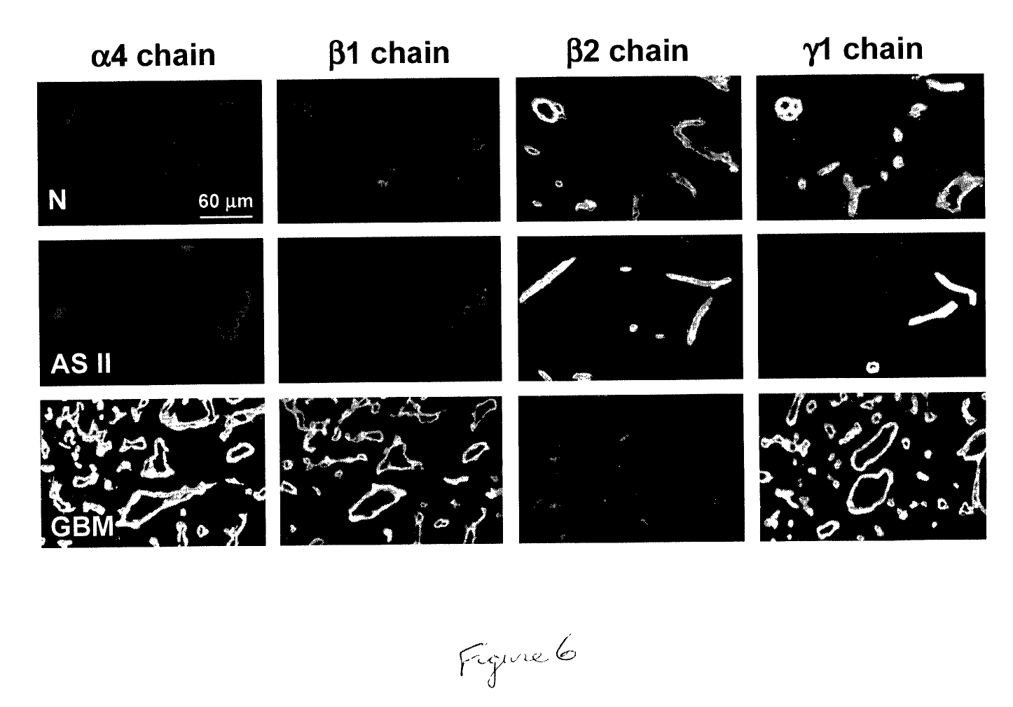

Laminin-8 has a subunit chain composition of α4β1γ1, and laminin-9 has component subunits α4β2γ1. Laminin α4 chain is also a constituent of recently described laminin-14 (α4β2γ3; Libby et al. [2000]). Therefore, it was determined which α4-containing laminins were predominantly expressed in normal brain tissue and in brain tumors. The expression of laminin-14 could not be demonstrated at the protein level since laminin α4 polypeptide chain was detected in GBM tissue (FIG. 6), while γ3 chain was never detected there (data not shown). At the same time, all constituent subunits of laminin-8 and laminin-9 were found in blood vessels of normal brain tissue and brain tumors.

The distribution of laminin-8 and laminin-9 subunit polypeptide chains in brain tumors was analyzed using samples from 12 gliomas (9 GBMs, one astrocytoma grade III and two astrocytomas grade II). Two benign meningiomas and two samples from normal brain tissue (obtained from trauma patients) were also analyzed.

Laminin α4 chain immunostaining was insignificant or weak in in blood vessel walls of normal brain and benign meningioma tissues. (FIG. 6). In astrocytoma grades II and III, laminin α4 expression was higher (FIG. 6; Table 7). All three astrocytoma cases studied showed increased staining compared to normal or meningioma tissue (p<0.03). In blood vessels of all GBMs and GBM-adjacent tissue, immunostaining for laminin α4 chain was generally much stronger (p<0.002). These results were in complete accordance with the gene array analysis and RT-PCR (Table 3, FIG. 3, and FIG. 5). Expression of laminin α4 subunit polypeptide has not been previously reported in connection with any human tumor.

The immunostaining patterns for other laminin subunits consistent with laminin α4 subunit-containing laminins were also determined using a panel of non-commercial antibodies targeted to various laminin subunit chains. Anti-γ1 subunit antibodies brightly stained blood vessel walls in all samples of normal brain, benign meningioma and malignant brain tumors (FIG. 6, Table 7). Beta (β)1 chain was weak in normal brain, two of three low grade astrocytomas, and three of nine GBMs (Table 7). In low-grade astrocytomas, laminin α4 staining strengthened compared to normal and meningioma tissue (FIG. 6; Table 7), but, as in normal tissue, there was stronger staining for laminin β2 chain than for laminin β1 chain. In all these cases, i.e., normal, meningioma, or low grade astrocytoma tissue, distinct to strong staining for β2 chain was observed (FIG. 6; Table 7), consistent with the predominant expression of laminin-9.

In GBMs, expression of laminin α4 became much stronger relative to normal, meningioma, or low grade astrocytoma tissues (FIG. 5, FIG. 6, and Table 7). However, in six of nine GBMs and in two of three GBM-adjacent tissue samples, immunostaining of laminin β1 chain was significantly stronger than for normal brain tissue, and stronger than immunostaining of laminin β2 subunit in the same samples (FIG. 6 and Table 7). There was no increase in expression of laminin β2 chain relative to normal tissue (FIG. 6 and Table 7). This result implied upregulation of laminin-8 in two thirds of GBM and tumor-adjacent tissues (FIG. 6).

Immunofluorescent staining was also performed with antibodies to other known laminin α chains, i.e., α1, α2, α3, and α5, which were expressed, except laminin α3 chain, which was always negative. The expression of other laminin a chains, did not differ significantly among any of the tissues examined. Thus, the distribution of laminin α1, α2, and α5 chains did not show significant difference among normal and malignant brain tissues (Table 8). However, laminin α2 chain was not found in meningiomas (benign tumors), while all other tissue samples were positively immunostained for laminin α2 subunit. (Table 8). Similar results were previously obtained for α1 and α2 chains in brain tumors. (Kulla, A. et al., Tenascin expression patterns and cells of monocyte lineage: relationship in human gliomas, Mod. Pathol. 13:56-67 [2000]). Taken together, these results make it unlikely that laminin isoforms other than laminin-8 became overexpressed in the neovasculature of the malignant tumors.

In summary, normal brain tissue and low-grade gliomas seemed to express relatively low levels of laminin α4, primarily as a constituent of laminin-9, whereas in the majority of GBMs, strong immunostaining was seen for laminin α4 subunit, as a constituent of laminin-8. Clinically, six out of six patients with high laminin α4 subunit expression (e.g., Patient Nos. 22, 39, 42, 45, 49, and 54) developed recurrences of glioma or died within two to six months (4.25±0.51 months, mean±SEM) after resection surgery, whereas all patients with intermediate laminin-8 expression (e.g., Patient Nos. 47, 50, 51) were diagnosed with recurrent tumors eight to eleven months (9.70±0.91 months, mean±SEM) after surgery. This difference was very significant, p=0.0007. Thus, an intermediate level of expression of laminin α4 subunit in samples from Patient Nos. 47, 50 and 51 corresponded to a relatively slow process of growth for these particular tumors. Together, these data demonstrate that a high level of expression of laminin α4 subunit, as a constituent of laminin-8 expression, is related to high tumor aggressiveness.

From Patient No. 39, primary tumor and adjacent tissue were examined, and it was determined that morphologically normal adjacent tissue expressed high levels of laminin α4 subunit, as a constituent of laminin-8. Based on data resulting from detection by methods of gene expression microarray, RT-PCR and immunostaining, a correlation was shown among the morphology of tumor-adjacent tissue, laminin α4 subunit distribution and recurrence of tumor development (Patient Nos. 16, 39, 47, and 49), demonstrating that overexpression of laminin α4 subunit in otherwise histologically normal tumor-adjacent tissue is predictive of tumor recurrence.

TABLE 7
Distribution of specific laminin chains in
human brain tumor tissue or tumor-adjacent
tissue (A) compared to normal brain tissue.^{a}

| | | α4 | β1 | | |
|---|---|---|---|---|---|
| Patient # | Diagnosis* | chain | chain | β2 chain | γl chain |
| 22 | GBM | +++ | ++++ | −/+ | +++ |
| 39 | GBM | +++ | +++ | + | +++ |
| 39-A** | Relatively normal | +++ | +++ | − | +++ |
| 42 | GBM | ++++ | +++ | ++ | +++ |
| 45 | GBM | ++++ | ++++ | + | ++++ |
| 47 | GBM | ++ | + | ++ | ++++ |
| 47-A | Focal invasion | +++ | +/++ | ++ | ++++ |
| 49 | Recurrent GBM | +++ | +++ | + | +++ |
| 49-A | Relatively normal | +/++ | + | +++ | +++ |
| 50 | GBM | ++/+++ | + | ++ | +++ |
| 51 | GBM | ++ | + | +++ | +++ |
| 54 | GBM | +++ | +++ | + | ++++ |
| 41 | Astrocytoma grade III | +/++ | + | ++ | +++ |
| 48 | Astrocytoma grade II | ++ | + | ++ | +++ |
| 53 | Astrocytoma grade II | ++ | ++ | +++ | +++ |
| 35 | Meningioma | + | ++ | + | +++ |
| 38 | Meningioma | −/+ | ++ | + | +++ |
| 40 | Normal brain | − | + | ++ | +++ |
| 44 | Normal brain | −/+ | + | ++ | +++ |

*Diagnosis based on direct histopathologic examination
**Tumor-adjacent tissue is obtained from the patient with the same code number.
^{a}Staining intensity grading is as follows: −, no staining; +, weak staining; ++ distinct staining; +++bright staining; ++++, very strong staining, /, some vessels in the same sample are in one category and some are in another category.

TABLE 8
Distribution of α1, α2 and α5 laminin chains in human brain tumors^{#}.

Sample

| # | Diagnosis | Sex/age | α1 chain | α2 chain | α5 chain |
|---|---|---|---|---|---|
| 22 | GBM | 58/M | +++ | +++ | +++ |
| 39* | GBM | 38/M | +++ | +++ | +++ |
| 39*- | Histologically | 38/M | +++ | +++ | +++ |
| adjacent | normal | | | | |
| 42 | GBM | 59/F | +++ | ++/+++ | +++ |
| 45 | GBM | 70/M | +++ | +++ | ++ |
| 47* | GBM | 57/M | ++ | +++ | ++/+++ |
| 47*- | Focal invasion | 57/M | +++ | +/++ | ++/+++ |
| adjacent | | | | | |
| 49* | GBM | 47/M | +++ | +++ | +++ |
| 49*- | Histologically | 47/M | +/++ | ++ | ++ |
| adjacent | normal | | | | |
| 50 | GBM | 61/M | ++/+++ | +++ | +++ |
| 51 | GBM | 79/M | +++ | +++ | +++ |
| 54 | GBM | 57/M | ++ | +++ | + |
| 41 | Astrocytoma | 27/M | +++ | +++ | +++ |
| | grade III | | | | |
| 48 | Astrocytoma | 28/M | +++ | +++ | +++ |
| | grade II | | | | |
| 53 | Astrocytoma | 32/M | +++ | +++ | +++ |
| | grade II | | | | |
| 35 | Meningioma | 53/F | ++ | − | +++ |
| 38 | Meningioma | 46/M | ++ | − | +++ |
| 40 | Normal brain | 44/F | ++/+++ | +++ | +++ |
| 44 | Normal brain | 47/M | +++ | +++ | +++ |

^{#}All studied samples were negative for laminin α3 chain. *, Tissue is obtained from the same patient. Staining intensity grading is as follows: −, no staining; +, weak staining; ++ distinct staining; +++bright staining; ++++, very strong staining, /, some vessels in the same sample are in one category and some are in another category.

We claim:

1. A method of detecting a malignant tumor in a human subject, comprising:
(a) collecting a sample of a bodily substance containing human nucleic acid or protein, said nucleic acid or protein having originated from cells of the human subject,
(b) detecting quantitatively or semi-quantitatively in the sample a level of expression for laminin α4 subunit protein or laminin α4-specific mRNA; and
(c) comparing the expression level in (b) to a level of expression in a normal control, wherein overexpression of laminin α4 subunit protein or laminin α4-specific mRNA, with respect to the control, indicates the presence of a malignant tumor in the human subject.

2. The method of claim 1, wherein the substance is blood, urine, lymph, cerebrospinal fluid, skin, stroma, vascular epithelium, oral epithelium, vaginal epithelium, cervical epithelium, uterine epithelium, intestinal epithelium, bronchial epithelium, esophageal epithelium, or mesothelium.

3. The method of claim 1, wherein the substance is a tissue sample.

4. The method of claim 3, wherein the tissue sample is collected from the brain of the subject.

5. The method of claim 3, wherein the tissue sample is a tumor tissue.

6. The method of claim 1, wherein the bodily substance is plasma.

7. The method of claim 1, wherein the bodily substance is a cellular material.

8. The method of claim 7, wherein the cellular material is derived from the human subject's brain kidney, bladder, ureter, urethra, thyroid, parotid gland, submaxillary gland, sublingual gland, lymph node, bone, cartilage, lung, mediastinum, breast, uterus, ovary, testis, prostate, cervix uteri, endometrium, pancreas, liver, spleen, adrenal, esophagus, stomach, or intestine.

9. The method of claim 1, wherein the neoplastic growth is a carcinoma, sarcoma, lymphoma, mesothelioma, melanoma, glioma, nephroblastoma, glioblastoma, oligodendroglioma, astrocytoma, ependymoma, primitive neuroectodermal tumor, atypical meningioma, malignant meningioma, or neuroblastoma.

10. The method of claim 1, wherein the hyperplastic and/or cytologically dysplastic cellular growth or proliferation is benign prostatic hyperplasia/dysplasia or cervical hyperplasia/dysplasia.

11. The method of claim 1, wherein the level of expression of laminin α4 subunit protein is detected.

12. The method of claim 1, wherein the level of expression of laminin α4-specific mRNA is detected.

13. The method of claim 12, wherein the expression level of laminin α4-specific mRNA is detected by measuring RNA.

14. The method of claim 12, wherein the expression level of laminin α4-specific mRNA is detected by measuring cDNA.

15. The method of claim 12, wherein a gene expression microarray is used to detect the level of expression of laminin α4-specific mRNA.

16. The method of claim 1, further comprising detecting the overexpression of laminin β1 subunit protein or laminin β1-specific mRNA relative to the normal control.

17. The method of claim 1, further comprising detecting quantitatively or semi-quantitatively in the sample a level of expression with respect to a normal control, of a gene encoding a protein selected from the group consisting of insulin-like growth factor binding protein precursor 3, transforming growth factor-β-induced gene, vascular endothelial growth factor, connective tissue growth factor, human insulin-like growth factor binding protein precursor 5, placental growth factor, transcription factor Ap-2, human insulin-like growth factor II, epidermal growth factor receptor, matrix metalloproteinase-2, keratin 18, vimentin, fibronectin 1, phospholipase A2 receptor, desmoplakin, tropomodulin, tenascin C, and collagen type IV α1 chain, or detecting a combination of expression levels for any of these.

18. A method of diagnosing the presence of a glioma in a human subject, comprising:
(a) obtaining a sample from the brain of the human subject;
(b) detecting quantitatively or semi-quantitatively in the sample a level of expression for laminin α4 subunit protein or laminin α4-specific mRNA; and
(c) comparing the expression level in (b) to a level of expression in a normal control, wherein overexpression of laminin α4 subunit protein or laminin α4-specific mRNA, with respect to the control, indicates the presence of glioma in the subject.

19. The method of claim 18, wherein the level of expression of laminin α4 subunit protein is detected.

20. The method of claim 18, wherein the level of expression of laminin α4-specific mRNA is detected.

21. The method of claim 20, wherein the expression level of laminin α4-specific mRNA is detected by measuring RNA.

22. The method of claim 20, wherein the expression level of laminin α4-specific mRNA is detected by measuring cDNA.

23. The method of claim 20, wherein a gene expression microarray is used to detect the level of expression of laminin α4-specific mRNA.

24. The method of claim 18, further comprising detecting the overexpression of laminin β1 subunit protein or laminin β1-specific mRNA relative to the normal control.

25. The method of claim 18, further comprising detecting quantitatively or semi-quantitatively in the sample a level of expression with respect to a normal control, of a gene encoding a protein selected from the group consisting of insulin-like growth factor binding protein precursor 3, transforming growth factor-β-induced gene, vascular endothelial growth factor, connective tissue growth factor, human insulin-like growth factor binding protein precursor 5, placental growth factor, transcription factor Ap-2, human insulin-like growth factor II, epidermal growth factor receptor, matrix metalloproteinase-2, keratin 18, vimentin, fibronectin 1, phospholipase A2 receptor, desmoplakin, tropomodulin, tenascin C, and collagen type IV α1 chain, or detecting a combination of expression levels for any of these.

26. The method of claim 18, wherein the sample is a tumor tissue.

27. The method of claim 18, wherein the sample comprises plasma.

28. A method of predicting the recurrence of a malignant tumor in a human subject from whom a tumor has been resected, comprising:
(a) obtaining a tissue sample from the human subject, said tissue sample being from a region adjacent to the site of the tumor;
(b) detecting quantitatively or semi-quantitatively a level of expression for laminin α4 subunit protein or laminin α4-specific mRNA in the sample; and
(c) comparing the expression level in (b) to a level of expression in a normal tissue control, wherein overexpression of laminin α4 subunit protein or laminin 14-specific mRNA, with respect to the control, is predictive of a recurrence of a malignant tumor in the subject.

29. The method of claim 28, wherein the tissue sample is histopathologically normal in appearance.

30. The method of claim 28, wherein the level of expression of laminin α4 subunit protein is detected.

31. The method of claim 28, wherein the level of expression of laminin 4-specific mRNA is detected.

32. The method of claim 31, wherein the expression level of laminin α4-specific mRNA is detected by measuring RNA.

33. The method of claim 31, wherein the expression level of laminin 4-specific mRNA is detected by measuring cDNA.

34. The method of claim 31, wherein a gene expression microarray is used to detect the level of expression of laminin α4-specific mRNA.

35. The method of claim 28, further comprising detecting quantitatively or semi-quantitatively in the sample a level of expression with respect to a normal tissue control, of a gene encoding a protein selected from the group consisting of insulin-like growth factor binding protein precursor 3, transforming growth factor-β-induced gene, vascular endothelial growth factor, connective tissue growth factor, human insulin-like growth factor binding protein precursor 5, placental growth factor, transcription factor Ap-2, human insulin-like growth factor II, epidermal growth factor receptor, matrix metalloproteinase-2, keratin 18, vimentin, fibronectin 1, phospholipase A2 receptor, desmoplakin, tropomodulin, tenascin C, and collagen type IV α1 chain, or detecting a combination of expression levels for any of these.

36. The method of claim 28, further comprising detecting the overexpression of laminin β1 subunit protein or laminin β1-specific nucleic acid relative to the normal tissue control.

37. The method of claim 28, wherein the level of expression of laminin α4 subunit protein is detected.

38. The method of claim 28, wherein the level of expression of laminin α4-specific mRNA is detected.

39. The method of claim 38, wherein the expression level of laminin α4-specific mRNA is detected by measuring RNA.

40. The method of claim 38, wherein the expression level of laminin α4-specific mRNA is detected by measuring cDNA.

41. The method of claim 38, wherein a gene expression microarray is used to detect the level of expression of laminin α4-specific mRNA.

42. The method of claim 28, further comprising detecting quantitatively or semi-quantitatively in the sample a level of expression with respect to a normal tissue control, of a gene encoding a protein selected from the group consisting of insulin-like growth factor binding protein precursor 3, transforming growth factor-β-induced gene, vascular endothelial growth factor, connective tissue growth factor, human insulin-like growth factor binding protein precursor 5, placental growth factor, transcription factor Ap-2, human insulin-like growth factor II, epidermal growth factor receptor, matrix metalloproteinase-2, keratin 18, vimentin, fibronectin 1, phospholipase A2 receptor, desmoplakin, tropomodulin, tenascin C, and collagen type IV α1 chain, or detecting a combination of expression levels for any of these.

43. The method of claim 28, further comprising detecting the overexpression of laminin β1 subunit protein or laminin β1-specific nucleic acid relative to the normal tissue control.

44. A method of predicting the recurrence of a glioma in a human subject from whom a glioma has been resected, comprising:
(a) obtaining a tissue sample from the brain of the human subject, said tissue sample being from a region adjacent to the site of the glioma;
(b) detecting quantitatively or semi-quantitatively a level of expression for laminin α4 subunit protein or laminin α4-specific mRNA in the sample; and
(c) comparing the expression level in (b) to a level of expression in a normal tissue control, wherein overexpression of laminin α4 subunit protein or laminin α4-specific mRNA, with respect to the control, is predictive of a recurrence of glioma in the subject.

45. The method of claim 44, wherein the tissue sample is histopathologically normal in appearance.

46. The method of claim 44, wherein the level of expression of laminin α4 subunit protein is detected.

47. The method of claim 44, wherein the level of expression of laminin α4-specific mRNA is detected.

48. The method of claim 47, wherein the expression level of laminin α4-specific mRNA is detected by measuring RNA.

49. The method of claim 47, wherein the expression level of laminin α4-specific mRNA is detected by measuring cDNA.

50. The method of claim 47, wherein a gene expression microarray is used to detect the level of expression of laminin α4-specific mRNA.

51. The method of claim 44, further comprising detecting quantitatively or semi-quantitatively in the sample a level of expression with respect to a normal tissue control, of a gene encoding a protein selected from the group consisting of insulin-like growth factor binding protein precursor 3, transforming growth factor-β-induced gene, vascular endothelial growth factor, connective tissue growth factor, human insulin-like growth factor binding protein precursor 5, placental growth factor, transcription factor Ap-2, human insulin-like growth factor II, epidermal growth factor receptor, matrix metalloproteinase-2, keratin 18, vimentin, fibronectin 1, phospholipase A2 receptor, desmoplakin, tropomodulin, tenascin C, and collagen type IV α1 chain, or detecting a combination of expression levels for any of these.

52. The method of claim 44, further comprising detecting the overexpression of laminin β1 subunit protein or laminin β1-specific nucleic acid relative to the normal tissue control.

53. A method of predicting recurrence of a glioma in a human subject from whom a glioma has been resected, comprising:
(a) obtaining a tissue sample from the brain of a human subject, said tissue sample being from a region adjacent to the site of the glioma, said sample comprising a cell expressing a plurality of mRNA species that are detectably distinct from one another;
(b) detecting quantitatively or semi-quantitatively an expression level for laminin α4-specific mRNA; and
(c) comparing the expression level in (b) to a level of expression in a normal tissue control, wherein overexpression of laminin α4-specific mRNA, with respect to the control, is predictive of a recurrence of glioma in the subject.

54. The method of claim 53, wherein a gene expression microarray is used to detect the level of expression of laminin α4-specific mRNA.

55. The method of claim 54, wherein the expression level of laminin α4-specific mRNA is detected by measuring RNA.

56. The method of claim 54, wherein the expression level of laminin α4-specific mRNA is detected by measuring cDNA.

57. The method of claim 53, further comprising detecting quantitatively or semi-quantitatively in the sample a level of expression with respect to a normal tissue control, of a growth factor-related gene encoding a protein selected from the group consisting of insulin-like growth factor binding protein precursor 3, transforming growth factor-β-induced gene, vascular endothelial growth factor, connective tissue growth factor, human insulin-like growth factor binding protein precursor 5, placental growth factor, transcription factor Ap-2, human insulin-like growth factor II, and epidermal growth factor receptor, whereby the relative aggressiveness of the glioma is predicted.

58. The method of claim 53, further comprising detecting quantitatively or semi-quantitatively in the sample a level of expression with respect to a normal tissue control, of a structural gene encoding a protein selected from the group consisting of matrix metalloproteinase-2, keratin 18, vimentin, fibronectin 1, phospholipase A2 receptor, desmoplakin, tropomodulin, tenascin C, and collagen type IV α1 chain, whereby the relative invasiveness of the glioma is predicted.

59. The method of claim 53, further comprising detecting the overexpression of laminin β1-specific mRNA relative to the normal tissue control.

60. A method of classifying the grade of a malignant tumor in a human subject, comprising:
(a) obtaining a tissue sample from the human subject, said sample comprising a cell expressing a plurality of mRNA species that are detectably distinct from one another;
(b) detecting quantitatively or semi-quantitatively an expression level for at least two of the plurality of mRNA species, wherein at least one of the detected mRNA species is a laminin α4-specific mRNA and at least one is specific to a growth factor-related gene or to a structural gene other than a laminin gene;
(c) constructing an expression profile of the sample comprising a combination of the detected expression levels of laminin α4-specific mRNA and the at least one other mRNA species specific to the growth factor-related gene or to the structural gene other than a laminin gene; and
(d) comparing the expression profile in (c) to an expression profile for a normal tissue control, wherein overexpression of laminin to4-specific mRNA, with respect to the control, is indicative of the presence and relatively high invasiveness of the tumor in the subject, wherein overexpression of the structural gene other than a laminin gene is indicative of relatively high tumor invasiveness, and wherein overexpression of the growth factor-related gene is indicative of relatively high tumor aggressiveness.

61. The method of claim 60, wherein the growth factor-related gene encodes a protein selected from the group consisting of insulin-like growth factor binding protein precursor 3, transforming growth factor-β-induced gene, vascular endothelial growth factor, connective tissue growth factor, human insulin-like growth factor binding protein precursor 5, placental growth factor, transcription factor Ap-2, human insulin-like growth factor II, and epidermal growth factor receptor.

62. The method of claim 60, wherein the structural gene encodes a protein selected from the group consisting of matrix metalloproteinase-2, keratin 18, vimentin, fibronectin 1, phospholipase A2 receptor, desmoplakin, tropomodulin, tenascin C, and collagen type IV α1 chain.

63. The method of claim 60, wherein the expression level of laminin α4-specific mRNA is detected by measuring RNA.

64. The method of claim 60, wherein the expression level of laminin α4-specific mRNA is detected by measuring cDNA.

65. The method of claim 60, wherein a gene expression microarray is used to detect the level of expression of laminin α4-specific mRNA.

66. The method of claim 60, further comprising detecting the overexpression of laminin β1-specific mRNA relative to the normal tissue control.

67. The method of claim 60, wherein the tissue sample is brain tissue.

68. The method of claim 60, wherein the tumor is a glial tumor.

69. A method of classifying the grade of a malignant tumor in a human subject, comprising:
(a) obtaining a tissue sample from the human subject, said sample comprising a cell expressing a plurality of protein species that are detectably distinct from one another;
(b) detecting quantitatively or semi-quantitatively an expression level for at least two of the plurality of protein species, wherein at least one of the detected protein species is a laminin α4 subunit protein and at least one is a growth factor-related protein or a structural protein other than a laminin protein,
(c) constructing an expression profile of the sample comprising a combination of the detected expression levels of laminin α4 subunit protein and the at least one other growth factor-related protein or the structural protein other than a laminin protein; and
(d) comparing the expression profile in (c) to an expression profile for a normal tissue control, wherein overexpression of laminin α4 subunit protein, with respect to the control, is indicative of the presence and relatively high invasiveness of a malignant tumoor in the subject, wherein overexpression of the structural protein other than a laminin protein is indicative of relatively high tumor invasiveness, and wherein overexpression of the growth factor-related protein is indicative of relatively high tumor aggressiveness.

70. The method of claim 69, wherein the growth factor-related protein is selected from the group consisting of insulin-like growth factor binding protein precursor 3, transforming growth factor-β-induced gene, vascular endothelial growth factor, connective tissue growth factor, human insulin-like growth factor binding protein precursor 5, placental growth factor, transcription factor Ap-2, human insulin-like growth factor II, and epidermal growth factor receptor.

71. The method of claim 69, wherein the structural protein is selected from the group consisting of matrix metalloproteinase-2, keratin 18, vimentin, fibronectin 1, phospholipase A2 receptor, desmoplakin, tropomodulin, tenascin C, and collagen type IV α1 chain.

72. The method of claim 69, further comprising detecting the overexpression of laminin β1 subunit protein relative to the normal tissue control.

73. The method of claim 69, wherein the tissue sample is brain tissue.

74. The method of claim 69, wherein the tumor is a glial tumor.
